(19) United States
(12) Patent Application Publication
(10) Pub. No.: US 2018/0144815 A1
(43) Pub. Date: May 24, 2018

(54) COMPUTER SYSTEM TO IDENTIFY ANOMALIES BASED ON COMPUTER GENERATED RESULTS

(71) Applicant: SAS Institute Inc., Cary, NC (US)

(72) Inventors: Emily Chapman-McQuiston, Cary, NC (US); Julius King, Apex, NC (US); Wallace Benjamin Wolverton, Raleigh, NC (US)

(73) Assignee: SAS Institute Inc., Cary, NC (US)

(21) Appl. No.: 15/819,562

(22) Filed: Nov. 21, 2017

Related U.S. Application Data
(60) Provisional application No. 62/426,026, filed on Nov. 23, 2016.

(51) Int. Cl. G16H 10/60

(52) U.S. Cl. CPC G16H 10/60

(57) ABSTRACT

One or more embodiments may include techniques to determine timeframes for target variables based on the scoring of data utilizing one or more models. Moreover, embodiments may include generating a first model based on a first subset of the data and a second model based on the second subset of the data, determining a first quality indication for the first model and a second quality indication for the second model, the first quality indication and the second quality indication based on one or more quality measurements, and the first quality indication and the second quality indication to indicate relative quality between the first model and the second model. Embodiments may include utilizing the first quality indication and the second quality indication to select the first model or the second model having higher quality, the selected first model or second model to score the data to determine the timeframes.

### RELATED APPLICATION

This application claims the benefit of priority of 35 U.S.C. § 119(e) to U.S. Provisional Patent Application Ser. No. 62/426,026, filed on Nov. 23, 2016, which is incorporated by reference.

### SUMMARY

This summary is not intended to identify only key or essential features of the described subject matter, nor is it intended to be used in isolation to determine the scope of the described subject matter. The subject matter should be understood by reference to appropriate portions of the entire specification of this patent, any or all drawings, and each claim.

Various embodiments described herein may include an apparatus comprising processing circuitry, and memory to store instructions that, when executed by the processing circuitry, cause the processing circuitry to obtain patient data, the patient data comprising medical events, consideration received for medical events, length of stays for the medical events, and diagnosis related groups (DRGs) for the medical events; determine a first subset of the patient data having consideration received for a medical event in a percentile grouping; determine a second subset of the patient data having the consideration received in another percentile grouping; generate a first model based on the first subset of the patient data, the first model for use to determine expected length of stay ranges for each of one or more DRGs; generate a second model based on the second subset of the patient data, the second model for use to determine the expected length of stay ranges for each of one or more DRGs; determine a first quality indication for the first model and a second quality indication for the second model, the first quality indication and the second quality indication based on one or more quality measurements, and the first quality indication and the second quality indication to indicate relative quality between the first model and the second model; utilize the first quality indication and the second quality indication to select the first model or the second model having higher quality, the selected first model or second model to score the patient data; and determine the expected length of stay ranges for the DRGs of the patient data based on the scoring of the patient data utilizing the selected first model or the second model, each of the expected length of stay ranges having a lower confidence limit and an upper confidence limit.

In embodiments, the first and second quality indications based on one or more quality measurements comprising an Akaike Information Criterion-Corrected (AICc) measurement of the first model and the second model, output parameter estimates indicating DRGs having significance for the first model and the second model, a first count of predictions for the first model matching actual length of stays and a second count of predictions for the second model matching the actual length of stays.

In embodiments, the processing circuitry of the apparatus to generate a third model based on the patient data, the third model for use to determine the expected length of stay ranges for each of one or more DRGs; generate a third quality indication for the third model, the third quality indication based on one or more quality measurements of the third model; utilize the third quality indication to select one of the first model, the second model, and the third model having higher quality, the selected first model, second model, or third model to score the patient data; and determine the expected length of stay ranges for the DRGs of the patient data based on the scoring of the patient data utilizing the selected first model, the second model, or the third model.

In one or more embodiments, the processing circuitry of the apparatus to determine claims associated with length of stays outside of the expected length of stay ranges for each of the one or more DRGs.

In embodiments, the processing circuitry of the apparatus to identify outlier length of stays in the patient data; and remove patient data associated with the outlier length of stays from the first subset and the second subset prior to generating the first model and second model.

In embodiments, the processing circuitry of the apparatus to identify locale information for the patient data and generate the first model and the second model based on the locale information.

In one or more embodiments, the processing circuitry of the apparatus to perform a log10 transformation on each length of stay in each of the first subset and the second subset prior to generating the first model and the second model.

In embodiments, the processing circuitry of the apparatus to identify claims associated with a readmission within a period of time of a date of a current admission for each of the claims for use as a variable in generating the first model and the second model, and group correlated variables of the patient data into clusters to generate the first model and the second model.

In embodiments, wherein each of the lower confidence limits is a minimum number of days and each of the upper confidence limits a maximum number of days.

In embodiments, wherein the first model and the second model are generalized linear mixed models.

Various embodiments may include at least one non-transitory computer-readable storage medium comprising instructions that when executed cause processing circuitry to obtain patient data, the patient data comprising medical events, consideration received for medical events, length of stays for the medical events, and diagnosis related groups (DRGs) for the medical events; determine a first subset of the patient data having consideration received for a medical event in a percentile grouping; determine a second subset of the patient data having the consideration received in another percentile grouping; generate a first model based on the first subset of the patient data, the first model for use to determine expected length of stay ranges for each of one or more DRGs; generate a second model based on the second subset of the patient data, the second model for use to determine the expected length of stay ranges for each of one or more DRGs; determine a first quality indication for the first model and a second quality indication for the second model, the first quality indication and the second quality indication based on one or more quality measurements, and the first quality indication and the second quality indication to indicate relative quality between the first model and the second model; utilize the first quality indication and the second quality indication to select the first model or the second model having higher quality, the selected first model or second model to score the patient data; and determine the expected length of stay ranges for the DRGs of the patient data based on the scoring of the patient data utilizing the selected first model or the second model, each of the expected length of stay ranges having a lower confidence limit and an upper confidence limit.

Various embodiments may include at least one non-transitory computer-readable storage medium comprising instructions that when executed cause processing circuitry to generate a third model based on the patient data, the third model for use to determine the expected length of stay ranges for each of one or more DRGs; generate a third quality indication for the third model, the third quality indication based on one or more quality measurements of the third model; utilize the third quality indication to select one of the first model, the second model, and the third model having higher quality, the selected first model, second model, or third model to score the patient data; and determine the expected length of stay ranges for the DRGs of the patient data based on the scoring of the patient data utilizing the selected first model, the second model, or the third model.

Various embodiments may include at least one non-transitory computer-readable storage medium comprising instructions that when executed cause processing circuitry to determine claims associated with length of stays outside of the expected length of stay ranges for each of the one or more DRGs.

Various embodiments may include at least one non-transitory computer-readable storage medium comprising instructions that when executed cause processing circuitry to identify outlier length of stays in the patient data; and remove patient data associated with the outlier length of stays from the first subset and the second subset prior to generating the first model and second model.

One or more embodiments may include at least one non-transitory computer-readable storage medium comprising instructions that when executed cause processing circuitry to identify locale information for the patient data and generate the first model and the second model based on the locale information.

Various embodiments may include at least one non-transitory computer-readable storage medium comprising instructions that when executed cause processing circuitry to identify claims associated with a readmission within a period of time of a date of a current admission for each of the claims for use as a variable in generating the first model and the second model, and group correlated variables of the patient data into clusters to generate the first model and the second model.

Some embodiments described herein may include a computer-implemented method, comprising obtaining patient data, the patient data comprising medical events, consideration received for medical events, length of stays for the medical events, and diagnosis related groups (DRGs) for the medical events; determining a first subset of the patient data having consideration received for a medical event in a percentile grouping; determining a second subset of the patient data having the consideration received in another percentile grouping; generating a first model based on the first subset of the patient data, the first model for use to determine expected length of stay ranges for each of one or more DRGs; generating a second model based on the second subset of the patient data, the second model for use to determine the expected length of stay ranges for each of one or more DRGs; determining a first quality indication for the first model and a second quality indication for the second model, the first quality indication and the second quality indication based on one or more quality measurements, and the first quality indication and the second quality indication to indicate relative quality between the first model and the second model; utilizing the first quality indication and the second quality indication to select the first model or the second model having higher quality, the selected first model or second model to score the patient data; and determining the expected length of stay ranges for the DRGs of the patient data based on the scoring of the patient data utilizing the selected first model or the second model, each of the expected length of stay ranges having a lower confidence limit and an upper confidence limit.

One or more embodiments described herein may include a computer-implemented method, comprising generating a third model based on the patient data, the third model for use to determine the expected length of stay ranges for each of one or more DRGs; generating a third quality indication for the third model, the third quality indication based on one or more quality measurements of the third model; utilizing the third quality indication to select one of the first model, the second model, and the third model having higher quality, the selected first model, second model, or third model to score the patient data; and determining the expected length of stay ranges for the DRGs of the patient data based on the scoring of the patient data utilizing the selected first model, the second model, or the third model.

Some embodiments described herein may include a computer-implemented method, comprising determining claims associated with length of stays outside of the expected length of stay ranges for each of the one or more DRGs.

Some embodiments described herein may include a computer-implemented method, comprising identify outlier length of stays in the patient data; and removing patient data associated with the outlier length of stays from the first subset and the second subset prior to generating the first model and second model.

Some embodiments described herein may include a computer-implemented method, comprising identifying locale information for the patient data and generate the first model and the second model based on the locale information.

Some embodiments described herein may include a computer-implemented method, comprising performing a log10 transformation on each length of stay in each of the first subset and the second subset prior to generating the first model and the second model.

Some embodiments described herein may include a computer-implemented method, comprising identifying claims associated with a readmission within a period of time of a date of a current admission for each of the claims for use as a variable in generating the first model and the second model, and group correlated variables of the patient data into clusters to generate the first model and the second model.

### BRIEF DESCRIPTION OF THE DRAWINGS

Embodiments of this disclosure are illustrated by way of example and not by way of limitation, in the figures of the accompanying drawings in which like reference numerals refer to similar elements.

FIG. 1 illustrates a block diagram that illustrates the hardware components of a computing system, according to some embodiments of the present technology.

FIG. 2 illustrates an example network including an example set of devices communicating with each other over an exchange system and via a network, according to some embodiments of the present technology.

FIG. 3 illustrates a representation of a conceptual model of a communications protocol system, according to some embodiments of the present technology.

FIG. 4 illustrates a communications grid computing system including a variety of control and worker nodes, according to some embodiments of the present technology.

FIG. 5 illustrates a flow chart showing an example process for adjusting a communications grid or a work project in a communications grid after a failure of a node, according to some embodiments of the present technology.

FIG. 6 illustrates a portion of a communications grid computing system including a control node and a worker node, according to some embodiments of the present technology.

FIG. 7 illustrates a flow chart showing an example process for executing a data analysis or processing project, according to some embodiments of the present technology.

FIG. 8 illustrates a block diagram including components of an Event Stream Processing Engine (ESPE), according to embodiments of the present technology.

FIG. 9 illustrates a flow chart showing an example process including operations performed by an event stream processing engine, according to some embodiments of the present technology.

FIG. 10 illustrates an ESP system interfacing between a publishing device and multiple event subscribing devices, according to embodiments of the present technology.

FIG. 11 illustrates a flow chart showing an example process of generating and using a machine-learning model according to some aspects.

FIG. 12 illustrates an example machine-learning model based on a neural network.

FIGS. 13A/13B illustrate examples of a distributed processing system.

FIG. 14 illustrates an example of a logic flow to process data for modeling.

FIG. 15 illustrates an example of a logic flow to perform one or more transformations in data.

FIG. 16A/B illustrate examples of logic flows to generate one or more models to generate predictions for a target variable.

FIG. 17 illustrates an example of a logic flow to generate results based on the models.

FIGS. 18A/18B illustrate an example logic flow to generate predictions.

FIGS. 19A-19E illustrate processing flows to process data, generate models and predictions, and detect anomalies.

### DETAILED DESCRIPTION

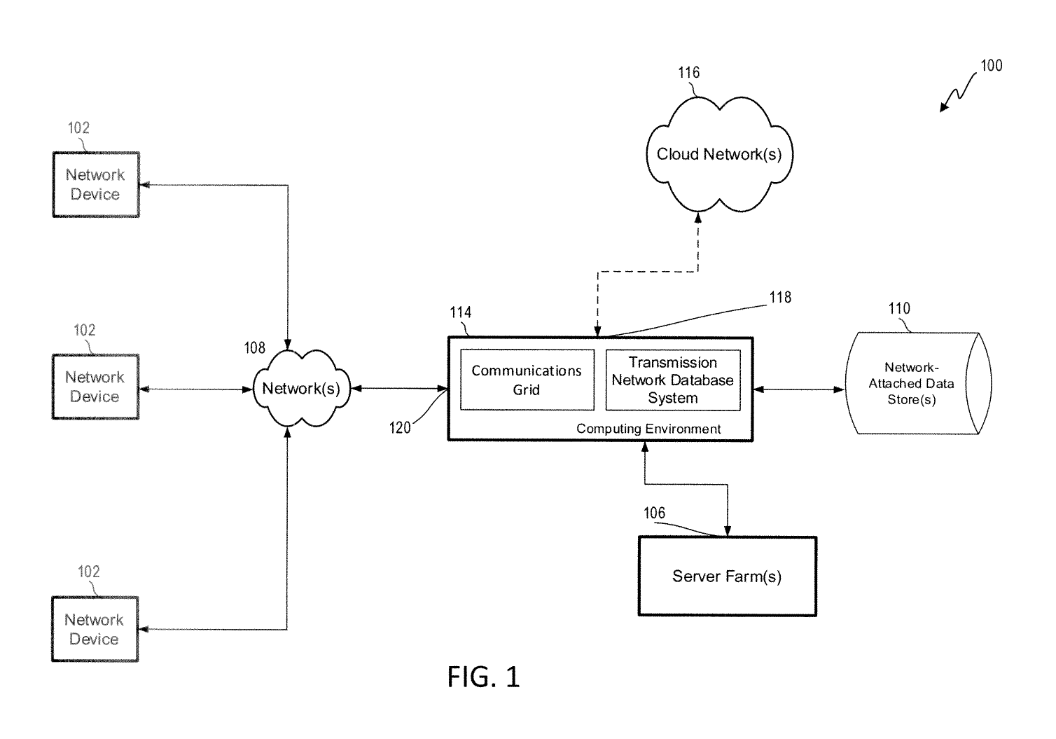

FIG. 1 is a block diagram that provides an illustration of the hardware components of a data transmission network 100, according to embodiments of the present technology. Data transmission network 100 is a specialized computer system that may be used for processing large amounts of data where a large number of computer processing cycles are required.

Data transmission network 100 may also include computing environment 114. Computing environment 114 may be a specialized computer or other machine that processes the data received within the data transmission network 100. Data transmission network 100 also includes one or more network devices 102. Network devices 102 may include client devices that attempt to communicate with computing environment 114. For example, network devices 102 may send data to the computing environment 114 to be processed, may send signals to the computing environment 114 to control different aspects of the computing environment or the data it is processing, among other reasons. Network devices 102 may interact with the computing environment 114 through a number of ways, such as, for example, over one or more networks 108. As shown in FIG. 1, computing environment 114 may include one or more other systems. For example, computing environment 114 may include a database system 118 and/or a communications grid 120.

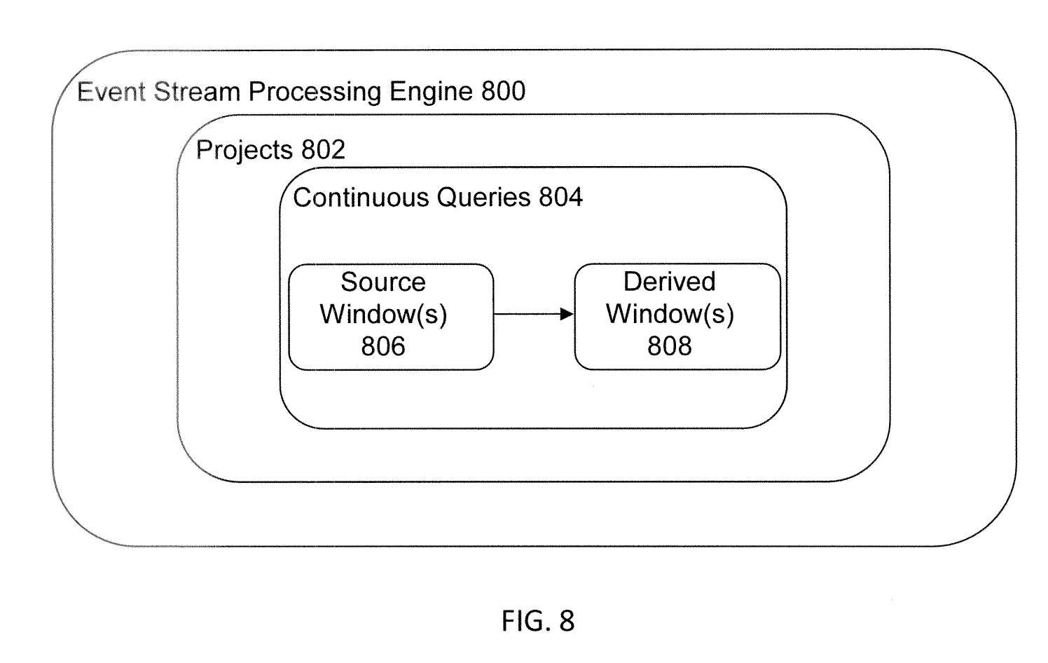

In other embodiments, network devices may provide a large amount of data, either all at once or streaming over a period of time (e.g., using event stream processing (ESP), described further with respect to FIGS. 8-10), to the computing environment 114 via networks 108. For example, network devices 102 may include network computers, sensors, databases, or other devices that may transmit or otherwise provide data to computing environment 114. For example, network devices may include local area network devices, such as routers, hubs, switches, or other computer networking devices. These devices may provide a variety of stored or generated data, such as network data or data specific to the network devices themselves. Network devices may also include sensors that monitor their environment or other devices to collect data regarding that environment or those devices, and such network devices may provide data they collect over time. Network devices may also include devices within the internet of things, such as devices within a home automation network. Some of these devices may be referred to as edge devices, and may involve edge computing circuitry. Data may be transmitted by network devices directly to computing environment 114 or to network-attached data stores, such as network-attached data stores 110 for storage so that the data may be retrieved later by the computing environment 114 or other portions of data transmission network 100.

Data transmission network 100 may also include one or more network-attached data stores 110. Network-attached data stores 110 are used to store data to be processed by the computing environment 114 as well as any intermediate or final data generated by the computing system in non-volatile memory. However in certain embodiments, the configuration of the computing environment 114 allows its operations to be performed such that intermediate and final data results can be stored solely in volatile memory (e.g., RAM), without a requirement that intermediate or final data results be stored to non-volatile types of memory (e.g., disk). This can be useful in certain situations, such as when the computing environment 114 receives ad hoc queries from a user and when responses, which are generated by processing large amounts of data, need to be generated on-the-fly. In this non-limiting situation, the computing environment 114 may be configured to retain the processed information within memory so that responses can be generated for the user at different levels of detail as well as allow a user to interactively query against this information.

Network-attached data stores may store a variety of different types of data organized in a variety of different ways and from a variety of different sources. For example, network-attached data storage may include storage other than primary storage located within computing environment 114 that is directly accessible by processors located therein. Network-attached data storage may include secondary, tertiary or auxiliary storage, such as large hard drives, servers, virtual memory, among other types. Storage devices may include portable or non-portable storage devices, optical storage devices, and various other mediums capable of storing, containing data. A machine-readable storage medium or computer-readable storage medium may include a non-transitory medium in which data can be stored and that does not include carrier waves and/or transitory electronic signals. Examples of a non-transitory medium may include, for example, a magnetic disk or tape, optical storage media such as compact disk or digital versatile disk, flash memory, memory or memory devices. A computer-program product may include code and/or machine-executable instructions that may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, among others. Furthermore, the data stores may hold a variety of different types of data. For example, network-attached data stores 110 may hold unstructured (e.g., raw) data, such as manufacturing data (e.g., a database containing records identifying products being manufactured with parameter data for each product, such as colors and models) or product sales databases (e.g., a database containing individual data records identifying details of individual product sales).

The unstructured data may be presented to the computing environment 114 in different forms such as a flat file or a conglomerate of data records, and may have data values and accompanying time stamps. The computing environment 114 may be used to analyze the unstructured data in a variety of ways to determine the best way to structure (e.g., hierarchically) that data, such that the structured data is tailored to a type of further analysis that a user wishes to perform on the data. For example, after being processed, the unstructured time stamped data may be aggregated by time (e.g., into daily time period units) to generate time series data and/or structured hierarchically according to one or more dimensions (e.g., parameters, attributes, and/or variables). For example, data may be stored in a hierarchical data structure, such as a ROLAP OR MOLAP database, or may be stored in another tabular form, such as in a flat-hierarchy form.

Data transmission network 100 may also include one or more server farms 106. Computing environment 114 may route select communications or data to the one or more sever farms 106 or one or more servers within the server farms. Server farms 106 can be configured to provide information in a predetermined manner For example, server farms 106 may access data to transmit in response to a communication. Server farms 106 may be separately housed from each other device within data transmission network 100, such as computing environment 114, and/or may be part of a device or system.

Server farms 106 may host a variety of different types of data processing as part of data transmission network 100. Server farms 106 may receive a variety of different data from network devices, from computing environment 114, from cloud network 116, or from other sources. The data may have been obtained or collected from one or more sensors, as inputs from a control database, or may have been received as inputs from an external system or device. Server farms 106 may assist in processing the data by turning raw data into processed data based on one or more rules implemented by the server farms. For example, sensor data may be analyzed to determine changes in an environment over time or in real-time.

Data transmission network 100 may also include one or more cloud networks 116. Cloud network 116 may include a cloud infrastructure system that provides cloud services. In certain embodiments, services provided by the cloud network 116 may include a host of services that are made available to users of the cloud infrastructure system on demand Cloud network 116 is shown in FIG. 1 as being connected to computing environment 114 (and therefore having computing environment 114 as its client or user), but cloud network 116 may be connected to or utilized by any of the devices in FIG. 1. Services provided by the cloud network can dynamically scale to meet the needs of its users. The cloud network 116 may comprise one or more computers, servers, and/or systems. In some embodiments, the computers, servers, and/or systems that make up the cloud network 116 are different from the user's own on-premises computers, servers, and/or systems. For example, the cloud network 116 may host an application, and a user may, via a communication network such as the Internet, on demand, order and use the application.

While each device, server and system in FIG. 1 is shown as a single device, it will be appreciated that multiple devices may instead be used. For example, a set of network devices can be used to transmit various communications from a single user, or remote server 140 may include a server stack. As another example, data may be processed as part of computing environment 114.

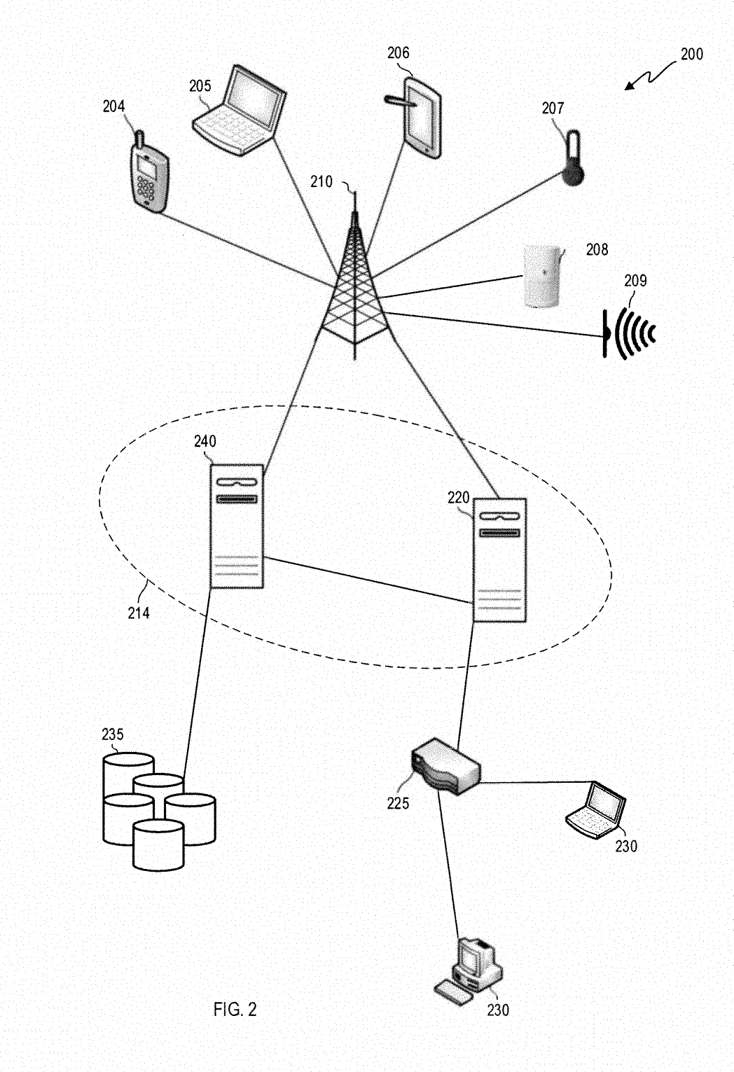

Each communication within data transmission network 100 (e.g., between client devices, between a device and connection management system 150, between servers 106 and computing environment 114 or between a server and a device) may occur over one or more networks 108. Networks 108 may include one or more of a variety of different types of networks, including a wireless network, a wired network, or a combination of a wired and wireless network. Examples of suitable networks include the Internet, a personal area network, a local area network (LAN), a wide area network (WAN), or a wireless local area network (WLAN). A wireless network may include a wireless interface or combination of wireless interfaces. As an example, a network in the one or more networks 108 may include a short-range communication channel, such as a Bluetooth or a Bluetooth Low Energy channel A wired network may include a wired interface. The wired and/or wireless networks may be implemented using routers, access points, bridges, gateways, or the like, to connect devices in the network 114, as will be further described with respect to FIG. 2. The one or more networks 108 can be incorporated entirely within or can include an intranet, an extranet, or a combination thereof. In one embodiment, communications between two or more systems and/or devices can be achieved by a secure communications protocol, such as secure sockets layer (SSL) or transport layer security (TLS). In addition, data and/or transactional details may be encrypted.

Some aspects may utilize the Internet of Things (IoT), where things (e.g., machines, devices, phones, sensors) can be connected to networks and the data from these things can be collected and processed within the things and/or external to the things. For example, the IoT can include sensors in many different devices, and high value analytics can be applied to identify hidden relationships and drive increased efficiencies. This can apply to both big data analytics and real-time (e.g., ESP) analytics. This will be described further below with respect to FIG. 2.

As noted, computing environment 114 may include a communications grid 120 and a transmission network database system 118. Communications grid 120 may be a grid-based computing system for processing large amounts of data. The transmission network database system 118 may be for managing, storing, and retrieving large amounts of data that are distributed to and stored in the one or more network-attached data stores 110 or other data stores that reside at different locations within the transmission network database system 118. The compute nodes in the grid-based computing system 120 and the transmission network database system 118 may share the same processor hardware, such as processors that are located within computing environment 114.

FIG. 2 illustrates an example network including an example set of devices communicating with each other over an exchange system and via a network, according to embodiments of the present technology. As noted, each communication within data transmission network 100 may occur over one or more networks. System 200 includes a network device 204 configured to communicate with a variety of types of client devices, for example client devices 230, over a variety of types of communication channels.

As shown in FIG. 2, network device 204 can transmit a communication over a network (e.g., a cellular network via a base station 210). The communication can be routed to another network device, such as network devices 205-209, via base station 210. The communication can also be routed to computing environment 214 via base station 210. For example, network device 204 may collect data either from its surrounding environment or from other network devices (such as network devices 205-209) and transmit that data to computing environment 214.

Although network devices 204-209 are shown in FIG. 2 as a mobile phone, laptop computer, tablet computer, temperature sensor, motion sensor, and audio sensor respectively, the network devices may be or include sensors that are sensitive to detecting aspects of their environment. For example, the network devices may include sensors such as water sensors, power sensors, electrical current sensors, chemical sensors, optical sensors, pressure sensors, geographic or position sensors (e.g., GPS), velocity sensors, acceleration sensors, flow rate sensors, among others. Examples of characteristics that may be sensed include force, torque, load, strain, position, temperature, air pressure, fluid flow, chemical properties, resistance, electromagnetic fields, radiation, irradiance, proximity, acoustics, moisture, distance, speed, vibrations, acceleration, electrical potential, electrical current, among others. The sensors may be mounted to various components used as part of a variety of different types of systems (e.g., an oil drilling operation). The network devices may detect and record data related to the environment that it monitors, and transmit that data to computing environment 214.

As noted, one type of system that may include various sensors that collect data to be processed and/or transmitted to a computing environment according to certain embodiments includes an oil drilling system. For example, the one or more drilling operation sensors may include surface sensors that measure a hook load, a fluid rate, a temperature and a density in and out of the wellbore, a standpipe pressure, a surface torque, a rotation speed of a drill pipe, a rate of penetration, a mechanical specific energy, etc. and downhole sensors that measure a rotation speed of a bit, fluid densities, downhole torque, downhole vibration (axial, tangential, lateral), a weight applied at a drill bit, an annular pressure, a differential pressure, an azimuth, an inclination, a dog leg severity, a measured depth, a vertical depth, a downhole temperature, etc. Besides the raw data collected directly by the sensors, other data may include parameters either developed by the sensors or assigned to the system by a client or other controlling device. For example, one or more drilling operation control parameters may control settings such as a mud motor speed to flow ratio, a bit diameter, a predicted formation top, seismic data, weather data, etc. Other data may be generated using physical models such as an earth model, a weather model, a seismic model, a bottom hole assembly model, a well plan model, an annular friction model, etc. In addition to sensor and control settings, predicted outputs, of for example, the rate of penetration, mechanical specific energy, hook load, flow in fluid rate, flow out fluid rate, pump pressure, surface torque, rotation speed of the drill pipe, annular pressure, annular friction pressure, annular temperature, equivalent circulating density, etc. may also be stored in the data warehouse.

In another example, another type of system that may include various sensors that collect data to be processed and/or transmitted to a computing environment according to certain embodiments includes a home automation or similar automated network in a different environment, such as an office space, school, public space, sports venue, or a variety of other locations. Network devices in such an automated network may include network devices that allow a user to access, control, and/or configure various home appliances located within the user's home (e.g., a television, radio, light, fan, humidifier, sensor, microwave, iron, and/or the like), or outside of the user's home (e.g., exterior motion sensors, exterior lighting, garage door openers, sprinkler systems, or the like). For example, network device 102 may include a home automation switch that may be coupled with a home appliance. In another embodiment, a network device can allow a user to access, control, and/or configure devices, such as office-related devices (e.g., copy machine, printer, or fax machine), audio and/or video related devices (e.g., a receiver, a speaker, a projector, a DVD player, or a television), media-playback devices (e.g., a compact disc player, a CD player, or the like), computing devices (e.g., a home computer, a laptop computer, a tablet, a personal digital assistant (PDA), a computing device, or a wearable device), lighting devices (e.g., a lamp or recessed lighting), devices associated with a security system, devices associated with an alarm system, devices that can be operated in an automobile (e.g., radio devices, navigation devices), and/or the like. Data may be collected from such various sensors in raw form, or data may be processed by the sensors to create parameters or other data either developed by the sensors based on the raw data or assigned to the system by a client or other controlling device.

In another example, another type of system that may include various sensors that collect data to be processed and/or transmitted to a computing environment according to certain embodiments includes a power or energy grid. A variety of different network devices may be included in an energy grid, such as various devices within one or more power plants, energy farms (e.g., wind farm, solar farm, among others) energy storage facilities, factories, homes and businesses of consumers, among others. One or more of such devices may include one or more sensors that detect energy gain or loss, electrical input or output or loss, and a variety of other efficiencies. These sensors may collect data to inform users of how the energy grid, and individual devices within the grid, may be functioning and how they may be made more efficient.

Network device sensors may also perform processing on data it collects before transmitting the data to the computing environment 114, or before deciding whether to transmit data to the computing environment 114. For example, network devices may determine whether data collected meets certain rules, for example by comparing data or values calculated from the data and comparing that data to one or more thresholds. The network device may use this data and/or comparisons to determine if the data should be transmitted to the computing environment 214 for further use or processing.

Computing environment 214 may include machines 220 and 240. Although computing environment 214 is shown in FIG. 2 as having two machines, 220 and 240, computing environment 214 may have only one machine or may have more than two machines. The machines that make up computing environment 214 may include specialized computers, servers, or other machines that are configured to individually and/or collectively process large amounts of data. The computing environment 214 may also include storage devices that include one or more databases of structured data, such as data organized in one or more hierarchies, or unstructured data. The databases may communicate with the processing devices within computing environment 214 to distribute data to them. Since network devices may transmit data to computing environment 214, that data may be received by the computing environment 214 and subsequently stored within those storage devices. Data used by computing environment 214 may also be stored in data stores 235, which may also be a part of or connected to computing environment 214.

Computing environment 214 can communicate with various devices via one or more routers 225 or other inter-network or intra-network connection components. For example, computing environment 214 may communicate with devices 230 via one or more routers 225. Computing environment 214 may collect, analyze and/or store data from or pertaining to communications, client device operations, client rules, and/or user-associated actions stored at one or more data stores 235. Such data may influence communication routing to the devices within computing environment 214, how data is stored or processed within computing environment 214, among other actions.

Notably, various other devices can further be used to influence communication routing and/or processing between devices within computing environment 214 and with devices outside of computing environment 214. For example, as shown in FIG. 2, computing environment 214 may include a web server 240. Thus, computing environment 214 can retrieve data of interest, such as client information (e.g., product information, client rules, etc.), technical product details, news, current or predicted weather, and so on.

In addition to computing environment 214 collecting data (e.g., as received from network devices, such as sensors, and client devices or other sources) to be processed as part of a big data analytics project, it may also receive data in real time as part of a streaming analytics environment. As noted, data may be collected using a variety of sources as communicated via different kinds of networks or locally. Such data may be received on a real-time streaming basis. For example, network devices may receive data periodically from network device sensors as the sensors continuously sense, monitor and track changes in their environments. Devices within computing environment 214 may also perform pre-analysis on data it receives to determine if the data received should be processed as part of an ongoing project. The data received and collected by computing environment 214, no matter what the source or method or timing of receipt, may be processed over a period of time for a client to determine results data based on the client's needs and rules.

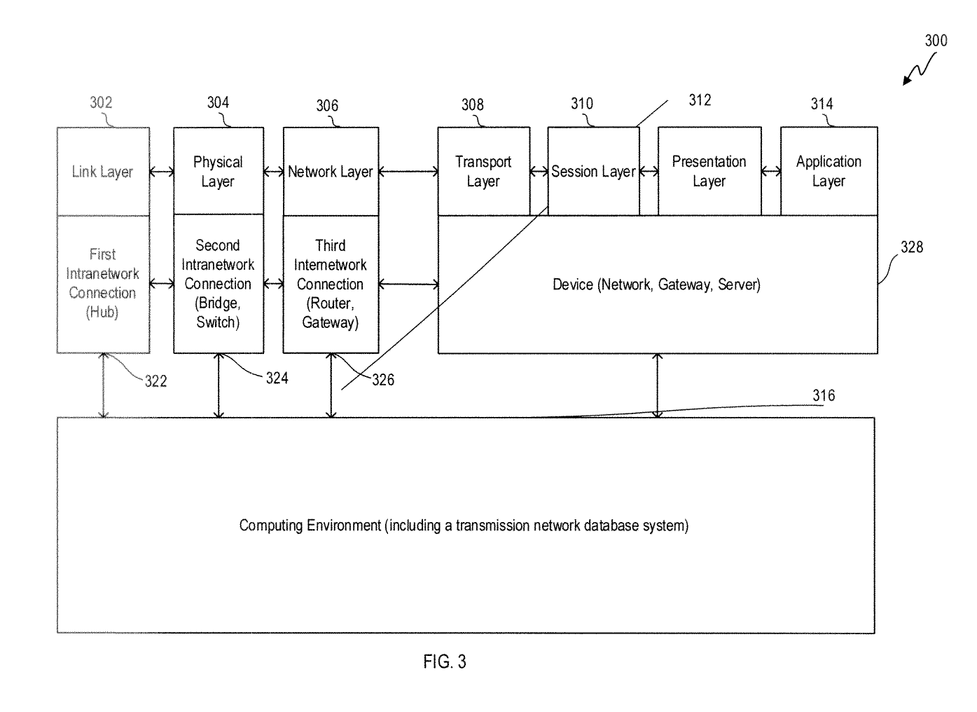

FIG. 3 illustrates a representation of a conceptual model of a communications protocol system, according to embodiments of the present technology. More specifically, FIG. 3 identifies operation of a computing environment in an Open Systems Interaction model that corresponds to various connection components. The model 300 shows, for example, how a computing environment, such as computing environment 314 (or computing environment 214 in FIG. 2) may communicate with other devices in its network, and control how communications between the computing environment and other devices are executed and under what conditions.

The model can include layers 302-314. The layers are arranged in a stack. Each layer in the stack serves the layer one level higher than it (except for the application layer, which is the highest layer), and is served by the layer one level below it (except for the physical layer, which is the lowest layer). The physical layer is the lowest layer because it receives and transmits raw bites of data, and is the farthest layer from the user in a communications system. On the other hand, the application layer is the highest layer because it interacts directly with a software application.

As noted, the model includes a physical layer 302. Physical layer 302 represents physical communication, and can define parameters of that physical communication. For example, such physical communication may come in the form of electrical, optical, or electromagnetic signals. Physical layer 302 also defines protocols that may control communications within a data transmission network.

Link layer 304 defines links and mechanisms used to transmit (i.e., move) data across a network. The link layer manages node-to-node communications, such as within a grid computing environment. Link layer 304 can detect and correct errors (e.g., transmission errors in the physical layer 302). Link layer 304 can also include a media access control (MAC) layer and logical link control (LLC) layer.

Network layer 306 defines the protocol for routing within a network. In other words, the network layer coordinates transferring data across nodes in a same network (e.g., such as a grid computing environment). Network layer 306 can also define the processes used to structure local addressing within the network.

Transport layer 308 can manage the transmission of data and the quality of the transmission and/or receipt of that data. Transport layer 308 can provide a protocol for transferring data, such as, for example, a Transmission Control Protocol (TCP). Transport layer 308 can assemble and disassemble data frames for transmission. The transport layer can also detect transmission errors occurring in the layers below it.

Session layer 310 can establish, maintain, and manage communication connections between devices on a network. In other words, the session layer controls the dialogues or nature of communications between network devices on the network. The session layer may also establish checkpointing, adjournment, termination, and restart procedures.

Presentation layer 312 can provide translation for communications between the application and network layers. In other words, this layer may encrypt, decrypt and/or format data based on data types known to be accepted by an application or network layer.

Application layer 314 interacts directly with software applications and end users , and manages communications between them. Application layer 314 can identify destinations, local resource states or availability and/or communication content or formatting using the applications.

Intra-network connection components 322 and 324 are shown to operate in lower levels, such as physical layer 302 and link layer 304, respectively. For example, a hub can operate in the physical layer, a switch can operate in the physical layer, and a router can operate in the network layer. Inter-network connection components 326 and 328 are shown to operate on higher levels, such as layers 306-314. For example, routers can operate in the network layer and network devices can operate in the transport, session, presentation, and application layers.

As noted, a computing environment 314 can interact with and/or operate on, in various embodiments, one, more, all or any of the various layers. For example, computing environment 314 can interact with a hub (e.g., via the link layer) so as to adjust which devices the hub communicates with. The physical layer may be served by the link layer, so it may implement such data from the link layer. For example, the computing environment 314 may control which devices it will receive data from. For example, if the computing environment 314 knows that a certain network device has turned off, broken, or otherwise become unavailable or unreliable, the computing environment 314 may instruct the hub to prevent any data from being transmitted to the computing environment 314 from that network device. Such a process may be beneficial to avoid receiving data that is inaccurate or that has been influenced by an uncontrolled environment. As another example, computing environment 314 can communicate with a bridge, switch, router or gateway and influence which device within the system (e.g., system 200) the component selects as a destination. In some embodiments, computing environment 314 can interact with various layers by exchanging communications with equipment operating on a particular layer by routing or modifying existing communications. In another embodiment, such as in a grid computing environment, a node may determine how data within the environment should be routed (e.g., which node should receive certain data) based on certain parameters or information provided by other layers within the model.

As noted, the computing environment 314 may be a part of a communications grid environment, the communications of which may be implemented as shown in the protocol of FIG. 3. For example, referring back to FIG. 2, one or more of machines 220 and 240 may be part of a communications grid computing environment. A gridded computing environment may be employed in a distributed system with non-interactive workloads where data resides in memory on the machines, or compute nodes. In such an environment, analytic code, instead of a database management system, controls the processing performed by the nodes. Data is co-located by pre-distributing it to the grid nodes, and the analytic code on each node loads the local data into memory. Each node may be assigned a particular task such as a portion of a processing project, or to organize or control other nodes within the grid.

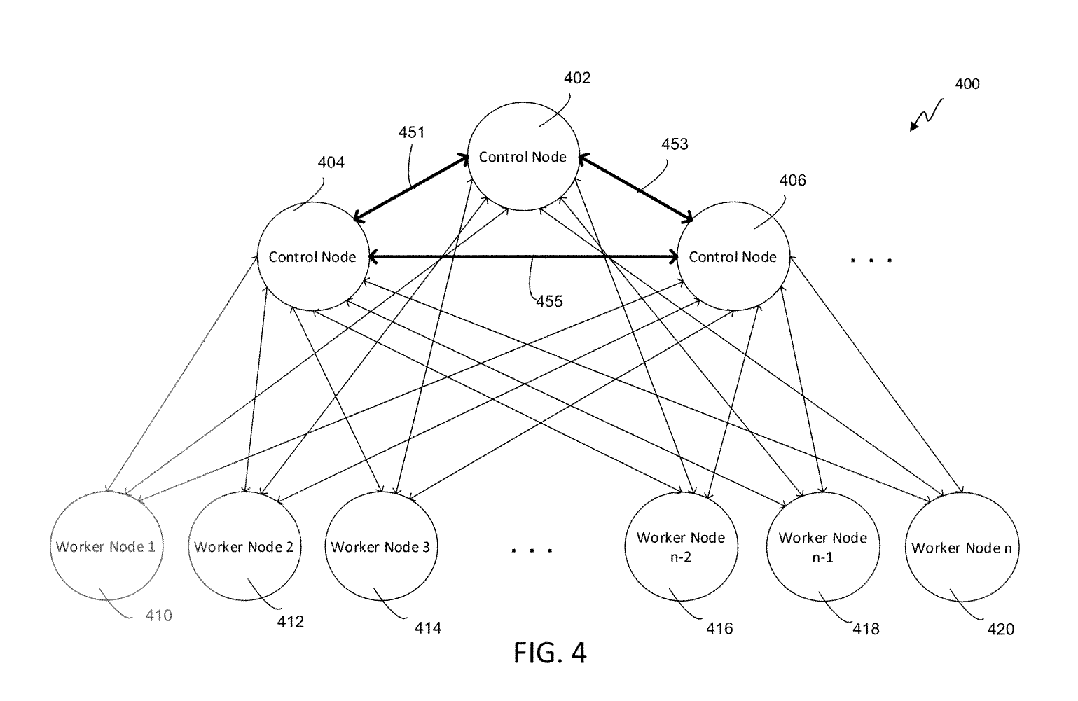

FIG. 4 illustrates a communications grid computing system 400 including a variety of control and worker nodes, according to embodiments of the present technology. Communications grid computing system 400 includes three control nodes and one or more worker nodes. Communications grid computing system 400 includes control nodes 402, 404, and 406. The control nodes are communicatively connected via communication paths 451, 453, and 455. Therefore, the control nodes may transmit information (e.g., related to the communications grid or notifications), to and receive information from each other. Although communications grid computing system 400 is shown in FIG. 4 as including three control nodes, the communications grid may include more or less than three control nodes.

Communications grid computing system (or just “communications grid”) 400 also includes one or more worker nodes. Shown in FIG. 4 are six worker nodes 410-420. Although FIG. 4 shows six worker nodes, a communications grid according to embodiments of the present technology may include more or less than six worker nodes. The number of worker nodes included in a communications grid may be dependent upon how large the project or data set is being processed by the communications grid, the capacity of each worker node, the time designated for the communications grid to complete the project, among others. Each worker node within the communications grid 400 may be connected (wired or wirelessly, and directly or indirectly) to control nodes 402-406. Therefore, each worker node may receive information from the control nodes (e.g., an instruction to perform work on a project) and may transmit information to the control nodes (e.g., a result from work performed on a project). Furthermore, worker nodes may communicate with each other (either directly or indirectly). For example, worker nodes may transmit data between each other related to a job being performed or an individual task within a job being performed by that worker node. However, in certain embodiments, worker nodes may not, for example, be connected (communicatively or otherwise) to certain other worker nodes. In an embodiment, worker nodes may only be able to communicate with the control node that controls it, and may not be able to communicate with other worker nodes in the communications grid, whether they are other worker nodes controlled by the control node that controls the worker node, or worker nodes that are controlled by other control nodes in the communications grid.

A control node may connect with an external device with which the control node may communicate (e.g., a grid user, such as a server or computer, may connect to a controller of the grid). For example, a server or computer may connect to control nodes and may transmit a project or job to the node. The project may include a data set. The data set may be of any size. Once the control node receives such a project including a large data set, the control node may distribute the data set or projects related to the data set to be performed by worker nodes. Alternatively, for a project including a large data set, the data set may be receive or stored by a machine other than a control node (e.g., a Hadoop data node).

Control nodes may maintain knowledge of the status of the nodes in the grid (i.e., grid status information), accept work requests from clients, subdivide the work across worker nodes, coordinate the worker nodes, among other responsibilities. Worker nodes may accept work requests from a control node and provide the control node with results of the work performed by the worker node. A grid may be started from a single node (e.g., a machine, computer, server, etc.). This first node may be assigned or may start as the primary control node that will control any additional nodes that enter the grid.

When a project is submitted for execution (e.g., by a client or a controller of the grid) it may be assigned to a set of nodes. After the nodes are assigned to a project, a data structure (i.e., a communicator) may be created. The communicator may be used by the project for information to be shared between the project code running on each node. A communication handle may be created on each node. A handle, for example, is a reference to the communicator that is valid within a single process on a single node, and the handle may be used when requesting communications between nodes.

A control node, such as control node 402, may be designated as the primary control node. A server, computer or other external device may connect to the primary control node. Once the control node receives a project, the primary control node may distribute portions of the project to its worker nodes for execution. For example, when a project is initiated on communications grid 400, primary control node 402 controls the work to be performed for the project in order to complete the project as requested or instructed. The primary control node may distribute work to the worker nodes based on various factors, such as which subsets or portions of projects may be completed most efficiently and in the correct amount of time. For example, a worker node may perform analysis on a portion of data that is already local (e.g., stored on) the worker node. The primary control node also coordinates and processes the results of the work performed by each worker node after each worker node executes and completes its job. For example, the primary control node may receive a result from one or more worker nodes, and the control node may organize (e.g., collect and assemble) the results received and compile them to produce a complete result for the project received from the end user.

Any remaining control nodes, such as control nodes 404 and 406, may be assigned as backup control nodes for the project. In an embodiment, backup control nodes may not control any portion of the project. Instead, backup control nodes may serve as a backup for the primary control node and take over as primary control node if the primary control node were to fail. If a communications grid were to include only a single control node, and the control node were to fail (e.g., the control node is shut off or breaks) then the communications grid as a whole may fail and any project or job being run on the communications grid may fail and may not complete. While the project may be run again, such a failure may cause a delay (severe delay in some cases, such as overnight delay) in completion of the project. Therefore, a grid with multiple control nodes, including a backup control node, may be beneficial.

To add another node or machine to the grid, the primary control node may open a pair of listening sockets, for example A socket may be used to accept work requests from clients, and the second socket may be used to accept connections from other grid nodes). The primary control node may be provided with a list of other nodes (e.g., other machines, computers, servers) that will participate in the grid, and the role that each node will fill in the grid. Upon startup of the primary control node (e.g., the first node on the grid), the primary control node may use a network protocol to start the server process on every other node in the grid. Command line parameters, for example, may inform each node of one or more pieces of information, such as: the role that the node will have in the grid, the host name of the primary control node, the port number on which the primary control node is accepting connections from peer nodes, among others. The information may also be provided in a configuration file, transmitted over a secure shell tunnel, recovered from a configuration server, among others. While the other machines in the grid may not initially know about the configuration of the grid, that information may also be sent to each other node by the primary control node. Updates of the grid information may also be subsequently sent to those nodes.

For any control node other than the primary control node added to the grid, the control node may open three sockets. The first socket may accept work requests from clients, the second socket may accept connections from other grid members, and the third socket may connect (e.g., permanently) to the primary control node. When a control node (e.g., primary control node) receives a connection from another control node, it first checks to see if the peer node is in the list of configured nodes in the grid. If it is not on the list, the control node may clear the connection. If it is on the list, it may then attempt to authenticate the connection. If authentication is successful, the authenticating node may transmit information to its peer, such as the port number on which a node is listening for connections, the host name of the node, information about how to authenticate the node, among other information. When a node, such as the new control node, receives information about another active node, it will check to see if it already has a connection to that other node. If it does not have a connection to that node, it may then establish a connection to that control node.

Any worker node added to the grid may establish a connection to the primary control node and any other control nodes on the grid. After establishing the connection, it may authenticate itself to the grid (e.g., any control nodes, including both primary and backup, or a server or user controlling the grid). After successful authentication, the worker node may accept configuration information from the control node.

When a node joins a communications grid (e.g., when the node is powered on or connected to an existing node on the grid or both), the node is assigned (e.g., by an operating system of the grid) a universally unique identifier (UUID). This unique identifier may help other nodes and external entities (devices, users, etc.) to identify the node and distinguish it from other nodes. When a node is connected to the grid, the node may share its unique identifier with the other nodes in the grid. Since each node may share its unique identifier, each node may know the unique identifier of every other node on the grid. Unique identifiers may also designate a hierarchy of each of the nodes (e.g., backup control nodes) within the grid. For example, the unique identifiers of each of the backup control nodes may be stored in a list of backup control nodes to indicate an order in which the backup control nodes will take over for a failed primary control node to become a new primary control node. However, a hierarchy of nodes may also be determined using methods other than using the unique identifiers of the nodes. For example, the hierarchy may be predetermined, or may be assigned based on other predetermined factors.

The grid may add new machines at any time (e.g., initiated from any control node). Upon adding a new node to the grid, the control node may first add the new node to its table of grid nodes. The control node may also then notify every other control node about the new node. The nodes receiving the notification may acknowledge that they have updated their configuration information.

Primary control node 402 may, for example, transmit one or more communications to backup control nodes 404 and 406 (and, for example, to other control or worker nodes within the communications grid). Such communications may sent periodically, at fixed time intervals, between known fixed stages of the project's execution, among other protocols. The communications transmitted by primary control node 402 may be of varied types and may include a variety of types of information. For example, primary control node 402 may transmit snapshots (e.g., status information) of the communications grid so that backup control node 404 always has a recent snapshot of the communications grid. The snapshot or grid status may include, for example, the structure of the grid (including, for example, the worker nodes in the grid, unique identifiers of the nodes, or their relationships with the primary control node) and the status of a project (including, for example, the status of each worker node's portion of the project). The snapshot may also include analysis or results received from worker nodes in the communications grid. The backup control nodes may receive and store the backup data received from the primary control node. The backup control nodes may transmit a request for such a snapshot (or other information) from the primary control node, or the primary control node may send such information periodically to the backup control nodes.

As noted, the backup data may allow the backup control node to take over as primary control node if the primary control node fails without requiring the grid to start the project over from scratch. If the primary control node fails, the backup control node that will take over as primary control node may retrieve the most recent version of the snapshot received from the primary control node and use the snapshot to continue the project from the stage of the project indicated by the backup data. This may prevent failure of the project as a whole.

A backup control node may use various methods to determine that the primary control node has failed. In one example of such a method, the primary control node may transmit (e.g., periodically) a communication to the backup control node that indicates that the primary control node is working and has not failed, such as a heartbeat communication. The backup control node may determine that the primary control node has failed if the backup control node has not received a heartbeat communication for a certain predetermined period of time. Alternatively, a backup control node may also receive a communication from the primary control node itself (before it failed) or from a worker node that the primary control node has failed, for example because the primary control node has failed to communicate with the worker node.

Different methods may be performed to determine which backup control node of a set of backup control nodes (e.g., backup control nodes 404 and 406) will take over for failed primary control node 402 and become the new primary control node. For example, the new primary control node may be chosen based on a ranking or “hierarchy” of backup control nodes based on their unique identifiers. In an alternative embodiment, a backup control node may be assigned to be the new primary control node by another device in the communications grid or from an external device (e.g., a system infrastructure or an end user, such as a server or computer, controlling the communications grid). In another alternative embodiment, the backup control node that takes over as the new primary control node may be designated based on bandwidth or other statistics about the communications grid.

A worker node within the communications grid may also fail. If a worker node fails, work being performed by the failed worker node may be redistributed amongst the operational worker nodes. In an alternative embodiment, the primary control node may transmit a communication to each of the operable worker nodes still on the communications grid that each of the worker nodes should purposefully fail also. After each of the worker nodes fail, they may each retrieve their most recent saved checkpoint of their status and re-start the project from that checkpoint to minimize lost progress on the project being executed.

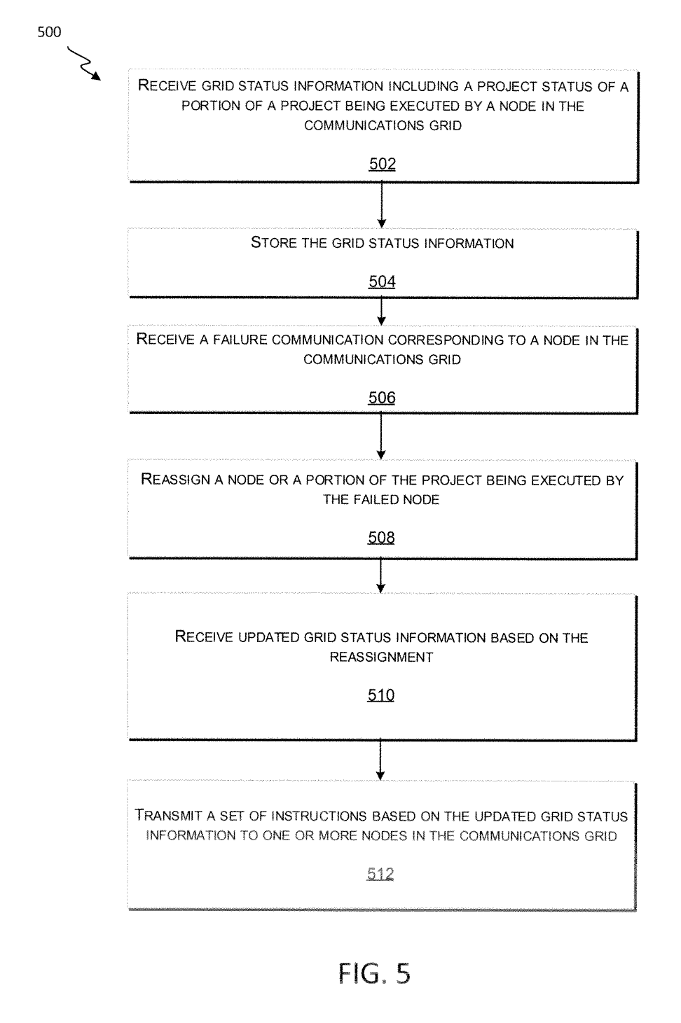

FIG. 5 illustrates a flow chart showing an example process for adjusting a communications grid or a work project in a communications grid after a failure of a node, according to embodiments of the present technology. The process may include, for example, receiving grid status information including a project status of a portion of a project being executed by a node in the communications grid, as described in operation 502. For example, a control node (e.g., a backup control node connected to a primary control node and a worker node on a communications grid) may receive grid status information, where the grid status information includes a project status of the primary control node or a project status of the worker node. The project status of the primary control node and the project status of the worker node may include a status of one or more portions of a project being executed by the primary and worker nodes in the communications grid. The process may also include storing the grid status information, as described in operation 504. For example, a control node (e.g., a backup control node) may store the received grid status information locally within the control node. Alternatively, the grid status information may be sent to another device for storage where the control node may have access to the information.

The process may also include receiving a failure communication corresponding to a node in the communications grid in operation 506. For example, a node may receive a failure communication including an indication that the primary control node has failed, prompting a backup control node to take over for the primary control node. In an alternative embodiment, a node may receive a failure that a worker node has failed, prompting a control node to reassign the work being performed by the worker node. The process may also include reassigning a node or a portion of the project being executed by the failed node, as described in operation 508. For example, a control node may designate the backup control node as a new primary control node based on the failure communication upon receiving the failure communication. If the failed node is a worker node, a control node may identify a project status of the failed worker node using the snapshot of the communications grid, where the project status of the failed worker node includes a status of a portion of the project being executed by the failed worker node at the failure time.

The process may also include receiving updated grid status information based on the reassignment, as described in operation 510, and transmitting a set of instructions based on the updated grid status information to one or more nodes in the communications grid, as described in operation 512. The updated grid status information may include an updated project status of the primary control node or an updated project status of the worker node. The updated information may be transmitted to the other nodes in the grid to update their stale stored information.

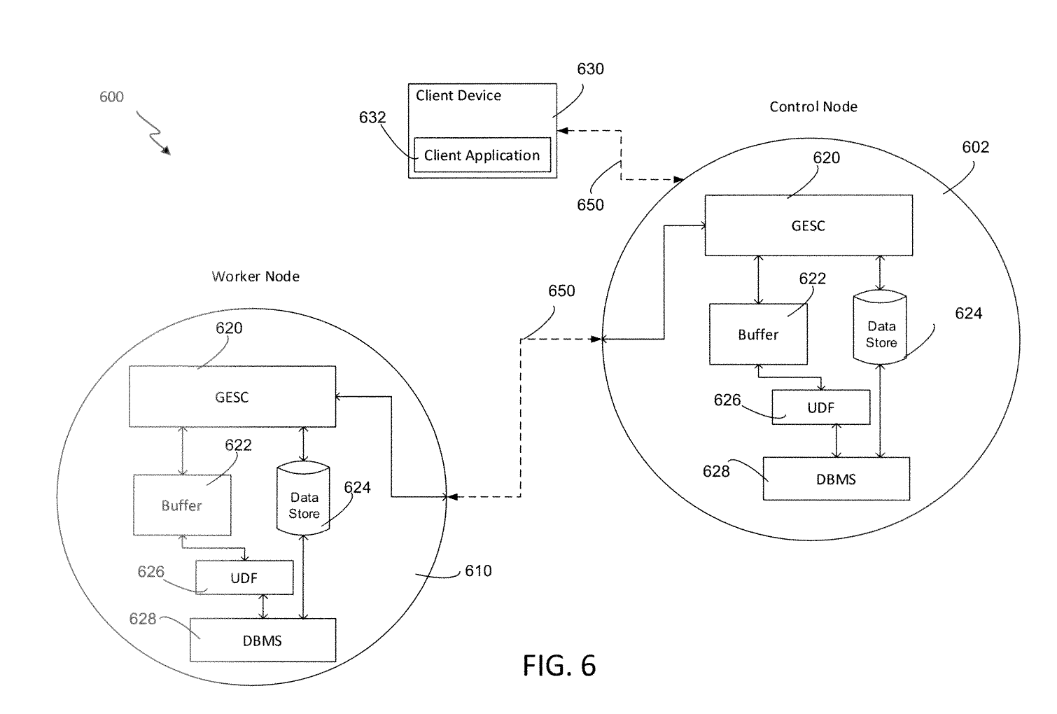

FIG. 6 illustrates a portion of a communications grid computing system 600 including a control node and a worker node, according to embodiments of the present technology. Communications grid 600 computing system includes one control node (control node 602) and one worker node (worker node 610) for purposes of illustration, but may include more worker and/or control nodes. The control node 602 is communicatively connected to worker node 610 via communication path 650. Therefore, control node 602 may transmit information (e.g., related to the communications grid or notifications), to and receive information from worker node 610 via path 650.

Similar to in FIG. 4, communications grid computing system (or just “communications grid”) 600 includes data processing nodes (control node 602 and worker node 610). Nodes 602 and 610 comprise multi-core data processors. Each node 602 and 610 includes a grid-enabled software component (GESC) 620 that executes on the data processor associated with that node and interfaces with buffer memory 622 also associated with that node. Each node 602 and 610 includes a database management software (DBMS) 628 that executes on a database server (not shown) at control node 602 and on a database server (not shown) at worker node 610.

Each node also includes a data store 624. Data stores 624, similar to network-attached data stores 110 in FIG. 1 and data stores 235 in FIG. 2, are used to store data to be processed by the nodes in the computing environment. Data stores 624 may also store any intermediate or final data generated by the computing system after being processed, for example in non-volatile memory. However in certain embodiments, the configuration of the grid computing environment allows its operations to be performed such that intermediate and final data results can be stored solely in volatile memory (e.g., RAM), without a requirement that intermediate or final data results be stored to non-volatile types of memory. Storing such data in volatile memory may be useful in certain situations, such as when the grid receives queries (e.g., ad hoc) from a client and when responses, which are generated by processing large amounts of data, need to be generated quickly or on-the-fly. In such a situation, the grid may be configured to retain the data within memory so that responses can be generated at different levels of detail and so that a client may interactively query against this information.

Each node also includes a user-defined function (UDF) 626. The UDF provides a mechanism for the DMBS 628 to transfer data to or receive data from the database stored in the data stores 624 that are managed by the DBMS. For example, UDF 626 can be invoked by the DBMS to provide data to the GESC for processing. The UDF 626 may establish a socket connection (not shown) with the GESC to transfer the data. Alternatively, the UDF 626 can transfer data to the GESC by writing data to shared memory accessible by both the UDF and the GESC.

The GESC 620 at the nodes 602 and 620 may be connected via a network, such as network 108 shown in FIG. 1. Therefore, nodes 602 and 620 can communicate with each other via the network using a predetermined communication protocol such as, for example, the Message Passing Interface (MPI). Each GESC 620 can engage in point-to-point communication with the GESC at another node or in collective communication with multiple GESCs via the network. The GESC 620 at each node may contain identical (or nearly identical) software instructions. Each node may be capable of operating as either a control node or a worker node. The GESC at the control node 602 can communicate, over a communication path 652, with a client deice 630. More specifically, control node 602 may communicate with client application 632 hosted by the client device 630 to receive queries and to respond to those queries after processing large amounts of data.

DMBS 628 may control the creation, maintenance, and use of database or data structure (not shown) within a nodes 602 or 610. The database may organize data stored in data stores 624. The DMBS 628 at control node 602 may accept requests for data and transfer the appropriate data for the request. With such a process, collections of data may be distributed across multiple physical locations. In this example, each node 602 and 610 stores a portion of the total data managed by the management system in its associated data store 624.

Furthermore, the DBMS may be responsible for protecting against data loss using replication techniques. Replication includes providing a backup copy of data stored on one node on one or more other nodes. Therefore, if one node fails, the data from the failed node can be recovered from a replicated copy residing at another node. However, as described herein with respect to FIG. 4, data or status information for each node in the communications grid may also be shared with each node on the grid.

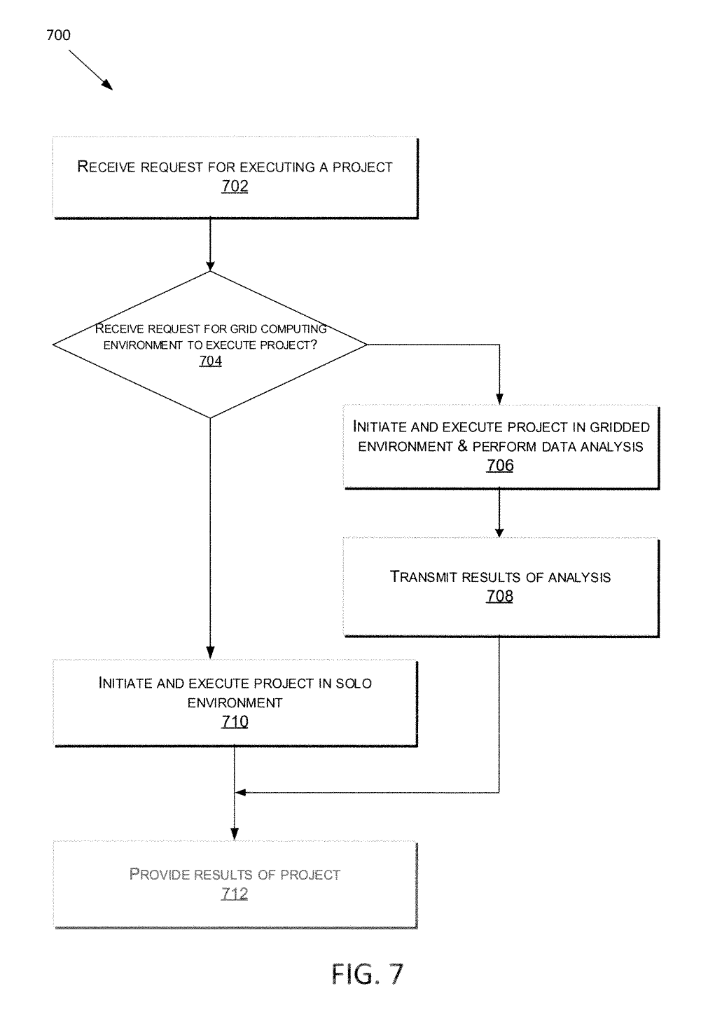

FIG. 7 illustrates a flow chart showing an example method for executing a project within a grid computing system, according to embodiments of the present technology. As described with respect to FIG. 6, the GESC at the control node may transmit data with a client device (e.g., client device 630) to receive queries for executing a project and to respond to those queries after large amounts of data have been processed. The query may be transmitted to the control node, where the query may include a request for executing a project, as described in operation 702. The query can contain instructions on the type of data analysis to be performed in the project and whether the project should be executed using the grid-based computing environment, as shown in operation 704.

To initiate the project, the control node may determine if the query requests use of the grid-based computing environment to execute the project. If the determination is no, then the control node initiates execution of the project in a solo environment (e.g., at the control node), as described in operation 710. If the determination is yes, the control node may initiate execution of the project in the grid-based computing environment, as described in operation 706. In such a situation, the request may include a requested configuration of the grid. For example, the request may include a number of control nodes and a number of worker nodes to be used in the grid when executing the project. After the project has been completed, the control node may transmit results of the analysis yielded by the grid, as described in operation 708. Whether the project is executed in a solo or grid-based environment, the control node provides the results of the project.

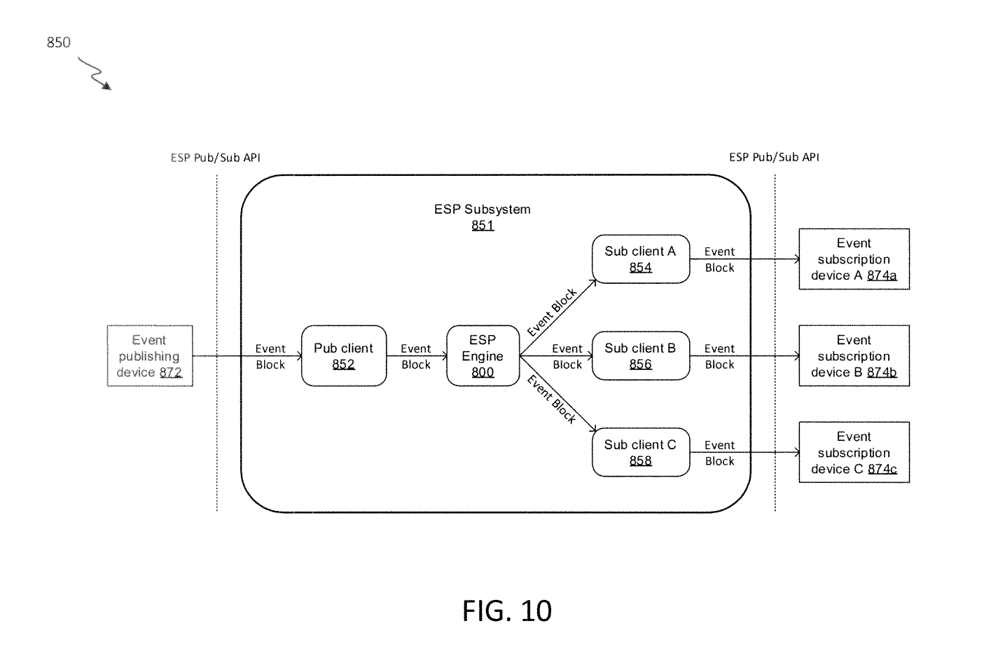

As noted with respect to FIG. 2, the computing environments described herein may collect data (e.g., as received from network devices, such as sensors, such as network devices 204-209 in FIG. 2, and client devices or other sources) to be processed as part of a data analytics project, and data may be received in real time as part of a streaming analytics environment (e.g., ESP). Data may be collected using a variety of sources as communicated via different kinds of networks or locally, such as on a real-time streaming basis. For example, network devices may receive data periodically from network device sensors as the sensors continuously sense, monitor and track changes in their environments. More specifically, an increasing number of distributed applications develop or produce continuously flowing data from distributed sources by applying queries to the data before distributing the data to geographically distributed recipients. An event stream processing engine (ESPE) may continuously apply the queries to the data as it is received and determines which entities should receive the data. Client or other devices may also subscribe to the ESPE or other devices processing ESP data so that they can receive data after processing, based on for example the entities determined by the processing engine. For example, client devices 230 in FIG. 2 may subscribe to the ESPE in computing environment 214. In another example, event subscription devices 874a-c, described further with respect to FIG. 10, may also subscribe to the ESPE. The ESPE may determine or define how input data or event streams from network devices or other publishers (e.g., network devices 204-209 in FIG. 2) are transformed into meaningful output data to be consumed by subscribers, such as for example client devices 230 in FIG. 2.

FIG. 8 illustrates a block diagram including components of an Event Stream Processing Engine (ESPE), according to embodiments of the present technology. ESPE 800 may include one or more projects 802. A project may be described as a second-level container in an engine model managed by ESPE 800 where a thread pool size for the project may be defined by a user. Each project of the one or more projects 802 may include one or more continuous queries 804 that contain data flows, which are data transformations of incoming event streams. The one or more continuous queries 804 may include one or more source windows 806 and one or more derived windows 808.

The ESPE may receive streaming data over a period of time related to certain events, such as events or other data sensed by one or more network devices. The ESPE may perform operations associated with processing data created by the one or more devices. For example, the ESPE may receive data from the one or more network devices 204-209 shown in FIG. 2. As noted, the network devices may include sensors that sense different aspects of their environments, and may collect data over time based on those sensed observations. For example, the ESPE may be implemented within one or more of machines 220 and 240 shown in FIG. 2. The ESPE may be implemented within such a machine by an ESP application. An ESP application may embed an ESPE with its own dedicated thread pool or pools into its application space where the main application thread can do application-specific work and the ESPE processes event streams at least by creating an instance of a model into processing objects.

The engine container is the top-level container in a model that manages the resources of the one or more projects 802. In an illustrative embodiment, for example, there may be only one ESPE 800 for each instance of the ESP application, and ESPE 800 may have a unique engine name Additionally, the one or more projects 802 may each have unique project names, and each query may have a unique continuous query name and begin with a uniquely named source window of the one or more source windows 806. ESPE 800 may or may not be persistent.

Continuous query modeling involves defining directed graphs of windows for event stream manipulation and transformation. A window in the context of event stream manipulation and transformation is a processing node in an event stream processing model. A window in a continuous query can perform aggregations, computations, pattern-matching, and other operations on data flowing through the window. A continuous query may be described as a directed graph of source, relational, pattern matching, and procedural windows. The one or more source windows 806 and the one or more derived windows 808 represent continuously executing queries that generate updates to a query result set as new event blocks stream through ESPE 800. A directed graph, for example, is a set of nodes connected by edges, where the edges have a direction associated with them.

An event object may be described as a packet of data accessible as a collection of fields, with at least one of the fields defined as a key or unique identifier (ID). The event object may be created using a variety of formats including binary, alphanumeric, XML, etc. Each event object may include one or more fields designated as a primary identifier (ID) for the event so ESPE 800 can support operation codes (opcodes) for events including insert, update, upsert, and delete. Upsert opcodes update the event if the key field already exists; otherwise, the event is inserted. For illustration, an event object may be a packed binary representation of a set of field values and include both metadata and field data associated with an event. The metadata may include an opcode indicating if the event represents an insert, update, delete, or upsert, a set of flags indicating if the event is a normal, partial-update, or a retention generated event from retention policy management, and a set of microsecond timestamps that can be used for latency measurements.

An event block object may be described as a grouping or package of event objects. An event stream may be described as a flow of event block objects. A continuous query of the one or more continuous queries 804 transforms a source event stream made up of streaming event block objects published into ESPE 800 into one or more output event streams using the one or more source windows 806 and the one or more derived windows 808. A continuous query can also be thought of as data flow modeling.

The one or more source windows 806 are at the top of the directed graph and have no windows feeding into them. Event streams are published into the one or more source windows 806, and from there, the event streams may be directed to the next set of connected windows as defined by the directed graph. The one or more derived windows 808 are all instantiated windows that are not source windows and that have other windows streaming events into them. The one or more derived windows 808 may perform computations or transformations on the incoming event streams. The one or more derived windows 808 transform event streams based on the window type (that is operators such as join, filter, compute, aggregate, copy, pattern match, procedural, union, etc.) and window settings. As event streams are published into ESPE 800, they are continuously queried, and the resulting sets of derived windows in these queries are continuously updated.

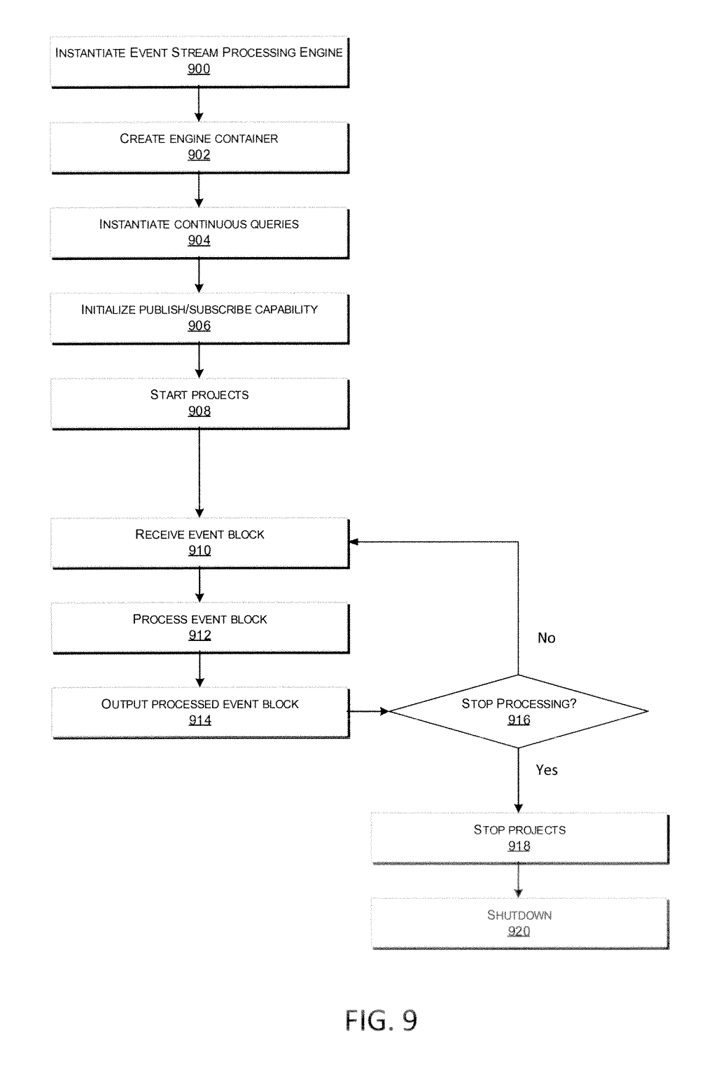

FIG. 9 illustrates a flow chart showing an example process including operations performed by an event stream processing engine, according to some embodiments of the present technology. As noted, the ESPE 800 (or an associated ESP application) defines how input event streams are transformed into meaningful output event streams. More specifically, the ESP application may define how input event streams from publishers (e.g., network devices providing sensed data) are transformed into meaningful output event streams consumed by subscribers (e.g., a data analytics project being executed by a machine or set of machines).

Within the application, a user may interact with one or more user interface windows presented to the user in a display under control of the ESPE independently or through a browser application in an order selectable by the user. For example, a user may execute an ESP application, which causes presentation of a first user interface window, which may include a plurality of menus and selectors such as drop down menus, buttons, text boxes, hyperlinks, etc. associated with the ESP application as understood by a person of skill in the art. As further understood by a person of skill in the art, various operations may be performed in parallel, for example, using a plurality of threads.

At operation 900, an ESP application may define and start an ESPE, thereby instantiating an ESPE at a device, such as machine 220 and/or 240. In an operation 902, the engine container is created. For illustration, ESPE 800 may be instantiated using a function call that specifies the engine container as a manager for the model.

In an operation 904, the one or more continuous queries 804 are instantiated by ESPE 800 as a model. The one or more continuous queries 804 may be instantiated with a dedicated thread pool or pools that generate updates as new events stream through ESPE 800. For illustration, the one or more continuous queries 804 may be created to model business processing logic within ESPE 800, to predict events within ESPE 800, to model a physical system within ESPE 800, to predict the physical system state within ESPE 800, etc. For example, as noted, ESPE 800 may be used to support sensor data monitoring and management (e.g., sensing may include force, torque, load, strain, position, temperature, air pressure, fluid flow, chemical properties, resistance, electromagnetic fields, radiation, irradiance, proximity, acoustics, moisture, distance, speed, vibrations, acceleration, electrical potential, or electrical current, etc.).

ESPE 800 may analyze and process events in motion or “event streams.” Instead of storing data and running queries against the stored data, ESPE 800 may store queries and stream data through them to allow continuous analysis of data as it is received. The one or more source windows 806 and the one or more derived windows 808 may be created based on the relational, pattern matching, and procedural algorithms that transform the input event streams into the output event streams to model, simulate, score, test, predict, etc. based on the continuous query model defined and application to the streamed data.

In an operation 906, a publish/subscribe (pub/sub) capability is initialized for ESPE 800. In an illustrative embodiment, a pub/sub capability is initialized for each project of the one or more projects 802. To initialize and enable pub/sub capability for ESPE 800, a port number may be provided. Pub/sub clients can use a host name of an ESP device running the ESPE and the port number to establish pub/sub connections to ESPE 800.

FIG. 10 illustrates an ESP system 850 interfacing between publishing device 872 and event subscribing devices 874a-c, according to embodiments of the present technology. ESP system 850 may include ESP device or subsystem 851, event publishing device 872, an event subscribing device A 874a, an event subscribing device B 874b, and an event subscribing device C 874c. Input event streams are output to ESP device 851 by publishing device 872. In alternative embodiments, the input event streams may be created by a plurality of publishing devices. The plurality of publishing devices further may publish event streams to other ESP devices. The one or more continuous queries instantiated by ESPE 800 may analyze and process the input event streams to form output event streams output to event subscribing device A 874a, event subscribing device B 874b, and event subscribing device C 874c. ESP system 850 may include a greater or a fewer number of event subscribing devices of event subscribing devices.

Publish-subscribe is a message-oriented interaction paradigm based on indirect addressing. Processed data recipients specify their interest in receiving information from ESPE 800 by subscribing to specific classes of events, while information sources publish events to ESPE 800 without directly addressing the receiving parties. ESPE 800 coordinates the interactions and processes the data. In some cases, the data source receives confirmation that the published information has been received by a data recipient.

A publish/subscribe API may be described as a library that enables an event publisher, such as publishing device 872, to publish event streams into ESPE 800 or an event subscriber, such as event subscribing device A 874a, event subscribing device B 874b, and event subscribing device C 874c, to subscribe to event streams from ESPE 800. For illustration, one or more publish/subscribe APIs may be defined. Using the publish/subscribe API, an event publishing application may publish event streams into a running event stream processor project source window of ESPE 800, and the event subscription application may subscribe to an event stream processor project source window of ESPE 800.

The publish/subscribe API provides cross-platform connectivity and endianness compatibility between ESP application and other networked applications, such as event publishing applications instantiated at publishing device 872, and event subscription applications instantiated at one or more of event subscribing device A 874a, event subscribing device B 874b, and event subscribing device C 874c.

Referring back to FIG. 9, operation 906 initializes the publish/subscribe capability of ESPE 800. In an operation 908, the one or more projects 802 are started. The one or more started projects may run in the background on an ESP device. In an operation 910, an event block object is received from one or more computing device of the event publishing device 872.

ESP subsystem 800 may include a publishing client 852, ESPE 800, a subscribing client A 854, a subscribing client B 856, and a subscribing client C 858. Publishing client 852 may be started by an event publishing application executing at publishing device 872 using the publish/subscribe API. Subscribing client A 854 may be started by an event subscription application A, executing at event subscribing device A 874a using the publish/subscribe API. Subscribing client B 856 may be started by an event subscription application B executing at event subscribing device B 874b using the publish/subscribe API. Subscribing client C 858 may be started by an event subscription application C executing at event subscribing device C 874c using the publish/subscribe API.

An event block object containing one or more event objects is injected into a source window of the one or more source windows 806 from an instance of an event publishing application on event publishing device 872. The event block object may generated, for example, by the event publishing application and may be received by publishing client 852. A unique ID may be maintained as the event block object is passed between the one or more source windows 806 and/or the one or more derived windows 808 of ESPE 800, and to subscribing client A 854, subscribing client B 806, and subscribing client C 808 and to event subscription device A 874a, event subscription device B 874b, and event subscription device C 874c. Publishing client 852 may further generate and include a unique embedded transaction ID in the event block object as the event block object is processed by a continuous query, as well as the unique ID that publishing device 872 assigned to the event block object.

In an operation 912, the event block object is processed through the one or more continuous queries 804. In an operation 914, the processed event block object is output to one or more computing devices of the event subscribing devices 874a-c. For example, subscribing client A 804, subscribing client B 806, and subscribing client C 808 may send the received event block object to event subscription device A 874a, event subscription device B 874b, and event subscription device C 874c, respectively.

ESPE 800 maintains the event block containership aspect of the received event blocks from when the event block is published into a source window and works its way through the directed graph defined by the one or more continuous queries 804 with the various event translations before being output to subscribers. Subscribers can correlate a group of subscribed events back to a group of published events by comparing the unique ID of the event block object that a publisher, such as publishing device 872, attached to the event block object with the event block ID received by the subscriber.

In an operation 916, a determination is made concerning whether or not processing is stopped. If processing is not stopped, processing continues in operation 910 to continue receiving the one or more event streams containing event block objects from the, for example, one or more network devices. If processing is stopped, processing continues in an operation 918. In operation 918, the started projects are stopped. In operation 920, the ESPE is shutdown.

As noted, in some embodiments, big data is processed for an analytics project after the data is received and stored. In other embodiments, distributed applications process continuously flowing data in real-time from distributed sources by applying queries to the data before distributing the data to geographically distributed recipients. As noted, an event stream processing engine (ESPE) may continuously apply the queries to the data as it is received and determines which entities receive the processed data. This allows for large amounts of data being received and/or collected in a variety of environments to be processed and distributed in real time. For example, as shown with respect to FIG. 2, data may be collected from network devices that may include devices within the internet of things, such as devices within a home automation network. However, such data may be collected from a variety of different resources in a variety of different environments. In any such situation, embodiments of the present technology allow for real-time processing of such data.

Aspects of the current disclosure provide technical solutions to technical problems, such as computing problems that arise when an ESP device fails which results in a complete service interruption and potentially significant data loss. The data loss can be catastrophic when the streamed data is supporting mission critical operations such as those in support of an ongoing manufacturing or drilling operation. An embodiment of an ESP system achieves a rapid and seamless failover of ESPE running at the plurality of ESP devices without service interruption or data loss, thus significantly improving the reliability of an operational system that relies on the live or real-time processing of the data streams. The event publishing systems, the event subscribing systems, and each ESPE not executing at a failed ESP device are not aware of or effected by the failed ESP device. The ESP system may include thousands of event publishing systems and event subscribing systems. The ESP system keeps the failover logic and awareness within the boundaries of out-messaging network connector and out-messaging network device.

In one example embodiment, a system is provided to support a failover when event stream processing (ESP) event blocks. The system includes, but is not limited to, an out-messaging network device and a computing device. The computing device includes, but is not limited to, a processor and a computer-readable medium operably coupled to the processor. The processor is configured to execute an ESP engine (ESPE). The computer-readable medium has instructions stored thereon that, when executed by the processor, cause the computing device to support the failover. An event block object is received from the ESPE that includes a unique identifier. A first status of the computing device as active or standby is determined. When the first status is active, a second status of the computing device as newly active or not newly active is determined. Newly active is determined when the computing device is switched from a standby status to an active status. When the second status is newly active, a last published event block object identifier that uniquely identifies a last published event block object is determined. A next event block object is selected from a non-transitory computer-readable medium accessible by the computing device. The next event block object has an event block object identifier that is greater than the determined last published event block object identifier. The selected next event block object is published to an out-messaging network device. When the second status of the computing device is not newly active, the received event block object is published to the out-messaging network device. When the first status of the computing device is standby, the received event block object is stored in the non-transitory computer-readable medium.

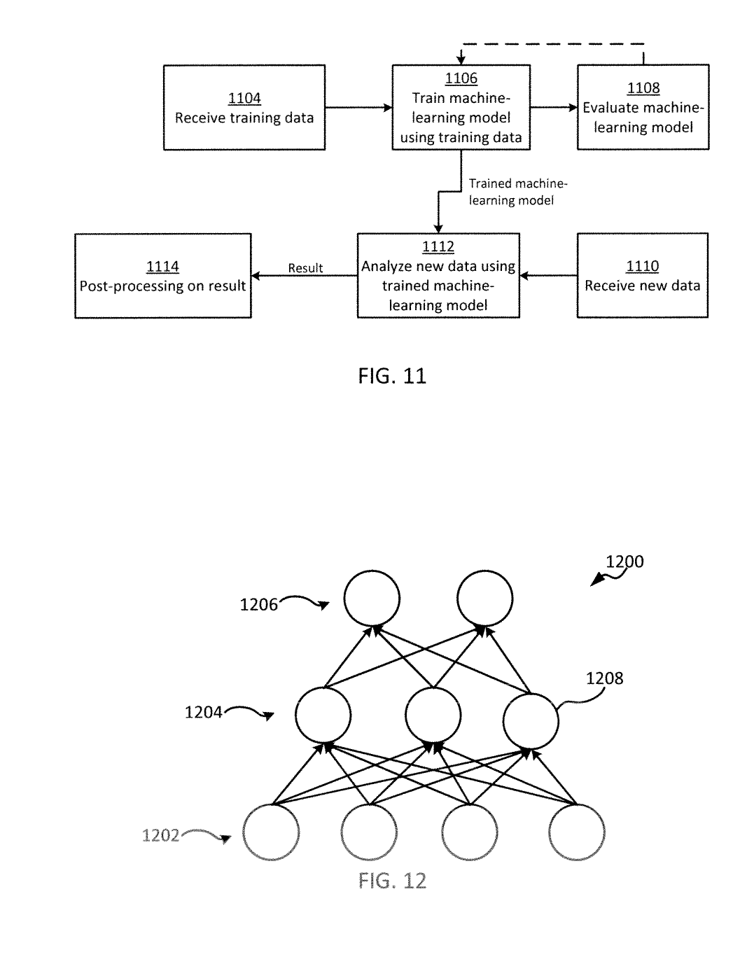

FIG. 11 is a flow chart of an example of a process for generating and using a machine-learning model according to some aspects. Machine learning is a branch of artificial intelligence that relates to mathematical models that can learn from, categorize, and make predictions about data. Such mathematical models, which can be referred to as machine-learning models, can classify input data among two or more classes; cluster input data among two or more groups; predict a result based on input data; identify patterns or trends in input data; identify a distribution of input data in a space; or any combination of these. Examples of machine-learning models can include (i) neural networks; (ii) decision trees, such as classification trees and regression trees; (iii) classifiers, such as Naïve bias classifiers, logistic regression classifiers, ridge regression classifiers, random forest classifiers, least absolute shrinkage and selector (LASSO) classifiers, and support vector machines; (iv) clusterers, such as k-means clusterers, mean-shift clusterers, and spectral clusterers; (v) factorizers, such as factorization machines, principal component analyzers and kernel principal component analyzers; and (vi) ensembles or other combinations of machine-learning models. In some examples, neural networks can include deep neural networks, feed-forward neural networks, recurrent neural networks, convolutional neural networks, radial basis function (RBF) neural networks, echo state neural networks, long short-term memory neural networks, bi-directional recurrent neural networks, gated neural networks, hierarchical recurrent neural networks, stochastic neural networks, modular neural networks, spiking neural networks, dynamic neural networks, cascading neural networks, neuro-fuzzy neural networks, or any combination of these.

Different machine-learning models may be used interchangeably to perform a task. Examples of tasks that can be performed at least partially using machine-learning models include various types of scoring; bioinformatics; cheminformatics; software engineering; fraud detection; customer segmentation; generating online recommendations; adaptive websites; determining customer lifetime value; search engines; placing advertisements in real time or near real time; classifying DNA sequences; affective computing; performing natural language processing and understanding; object recognition and computer vision; robotic locomotion; playing games; optimization and metaheuristics; detecting network intrusions; medical diagnosis and monitoring; or predicting when an asset, such as a machine, will need maintenance.

Any number and combination of tools can be used to create machine-learning models. Examples of tools for creating and managing machine-learning models can include SAS® Enterprise Miner, SAS® Rapid Predictive Modeler, and SAS® Model Manager, SAS Cloud Analytic Services (CAS)®, SAS Viya® of all which are by SAS Institute Inc. of Cary, N.C.

Machine-learning models can be constructed through an at least partially automated (e.g., with little or no human involvement) process called training. During training, input data can be iteratively supplied to a machine-learning model to enable the machine-learning model to identify patterns related to the input data or to identify relationships between the input data and output data. With training, the machine-learning model can be transformed from an untrained state to a trained state. Input data can be split into one or more training sets and one or more validation sets, and the training process may be repeated multiple times. The splitting may follow a k-fold cross-validation rule, a leave-one-out-rule, a leave-p-out rule, or a holdout rule. An overview of training and using a machine-learning model is described below with respect to the flow chart of FIG. 11.

In block 1104, training data is received. In some examples, the training data is received from a remote database or a local database, constructed from various subsets of data, or input by a user. The training data can be used in its raw form for training a machine-learning model or pre-processed into another form, which can then be used for training the machine-learning model. For example, the raw form of the training data can be smoothed, truncated, aggregated, clustered, or otherwise manipulated into another form, which can then be used for training the machine-learning model.

In block 1106, a machine-learning model is trained using the training data. The machine-learning model can be trained in a supervised, unsupervised, or semi-supervised manner In supervised training, each input in the training data is correlated to a desired output. This desired output may be a scalar, a vector, or a different type of data structure such as text or an image. This may enable the machine-learning model to learn a mapping between the inputs and desired outputs. In unsupervised training, the training data includes inputs, but not desired outputs, so that the machine-learning model has to find structure in the inputs on its own. In semi-supervised training, only some of the inputs in the training data are correlated to desired outputs.

In block 1108, the machine-learning model is evaluated. For example, an evaluation dataset can be obtained, for example, via user input or from a database. The evaluation dataset can include inputs correlated to desired outputs. The inputs can be provided to the machine-learning model and the outputs from the machine-learning model can be compared to the desired outputs. If the outputs from the machine-learning model closely correspond with the desired outputs, the machine-learning model may have a high degree of accuracy. For example, if 90% or more of the outputs from the machine-learning model are the same as the desired outputs in the evaluation dataset, the machine-learning model may have a high degree of accuracy. Otherwise, the machine-learning model may have a low degree of accuracy. The 90% number is an example only. A realistic and desirable accuracy percentage is dependent on the problem and the data.

In some examples, if the machine-learning model has an inadequate degree of accuracy for a particular task, the process can return to block 1106, where the machine-learning model can be further trained using additional training data or otherwise modified to improve accuracy. If the machine-learning model has an adequate degree of accuracy for the particular task, the process can continue to block 1110.

In block 1110, new data is received. In some examples, the new data is received from a remote database or a local database, constructed from various subsets of data, or input by a user. The new data may be unknown to the machine-learning model. For example, the machine-learning model may not have previously processed or analyzed the new data.

In block 1112, the trained machine-learning model is used to analyze the new data and provide a result. For example, the new data can be provided as input to the trained machine-learning model. The trained machine-learning model can analyze the new data and provide a result that includes a classification of the new data into a particular class, a clustering of the new data into a particular group, a prediction based on the new data, or any combination of these.

In block 1114, the result is post-processed. For example, the result can be added to, multiplied with, or otherwise combined with other data as part of a job. As another example, the result can be transformed from a first format, such as a time series format, into another format, such as a count series format. Any number and combination of operations can be performed on the result during post-processing.

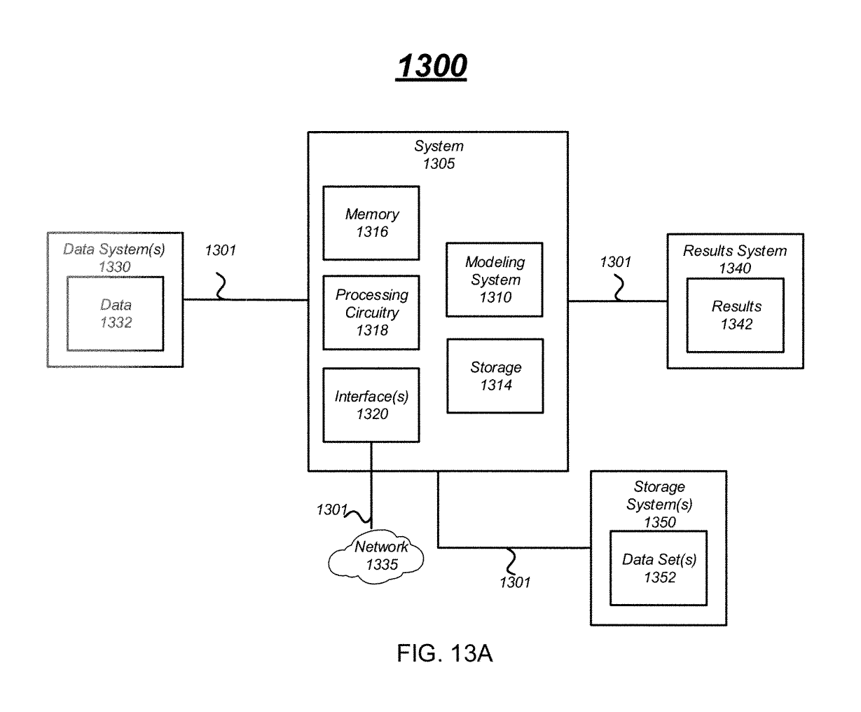

A more specific example of a machine-learning model is the neural network 1200 shown in FIG. 12. The neural network 1200 is represented as multiple layers of interconnected neurons, such as neuron 1208, that can exchange data between one another. The layers include an input layer 1202 for receiving input data, a hidden layer 1204, and an output layer 1206 for providing a result. The hidden layer 1204 is referred to as hidden because it may not be directly observable or have its input directly accessible during the normal functioning of the neural network 1200. Although the neural network 1200 is shown as having a specific number of layers and neurons for exemplary purposes, the neural network 1200 can have any number and combination of layers, and each layer can have any number and combination of neurons.

The neurons and connections between the neurons can have numeric weights, which can be tuned during training. For example, training data can be provided to the input layer 1202 of the neural network 1200, and the neural network 1200 can use the training data to tune one or more numeric weights of the neural network 1200. In some examples, the neural network 1200 can be trained using backpropagation. Backpropagation can include determining a gradient of a particular numeric weight based on a difference between an actual output of the neural network 1200 and a desired output of the neural network 1200. Based on the gradient, one or more numeric weights of the neural network 1200 can be updated to reduce the difference, thereby increasing the accuracy of the neural network 1200. This process can be repeated multiple times to train the neural network 1200. For example, this process can be repeated hundreds or thousands of times to train the neural network 1200.

In some examples, the neural network 1200 is a feed-forward neural network. In a feed-forward neural network, every neuron only propagates an output value to a subsequent layer of the neural network 1200. For example, data may only move one direction (forward) from one neuron to the next neuron in a feed-forward neural network.

In other examples, the neural network 1200 is a recurrent neural network. A recurrent neural network can include one or more feedback loops, allowing data to propagate in both forward and backward through the neural network 1200. This can allow for information to persist within the recurrent neural network. For example, a recurrent neural network can determine an output based at least partially on information that the recurrent neural network has seen before, giving the recurrent neural network the ability to use previous input to inform the output.

In some examples, the neural network 1200 operates by receiving a vector of numbers from one layer; transforming the vector of numbers into a new vector of numbers using a matrix of numeric weights, a nonlinearity, or both; and providing the new vector of numbers to a subsequent layer of the neural network 1200. Each subsequent layer of the neural network 1200 can repeat this process until the neural network 1200 outputs a final result at the output layer 1206. For example, the neural network 1200 can receive a vector of numbers as an input at the input layer 1202. The neural network 1200 can multiply the vector of numbers by a matrix of numeric weights to determine a weighted vector. The matrix of numeric weights can be tuned during the training of the neural network 1200. The neural network 1200 can transform the weighted vector using a nonlinearity, such as a sigmoid tangent or the hyperbolic tangent. In some examples, the nonlinearity can include a rectified linear unit, which can be expressed using the following equation:

y=max(x,0)

where y is the output and x is an input value from the weighted vector. The transformed output can be supplied to a subsequent layer, such as the hidden layer 1204, of the neural network 1200. The subsequent layer of the neural network 1200 can receive the transformed output, multiply the transformed output by a matrix of numeric weights and a nonlinearity, and provide the result to yet another layer of the neural network 1200. This process continues until the neural network 1200 outputs a final result at the output layer 1206.

Other examples of the present disclosure may include any number and combination of machine-learning models having any number and combination of characteristics. The machine-learning model(s) can be trained in a supervised, semi-supervised, or unsupervised manner, or any combination of these. The machine-learning model(s) can be implemented using a single computing device or multiple computing devices, such as the communications grid computing system 400 discussed above.

Implementing some examples of the present disclosure at least in part by using machine-learning models can reduce the total number of processing iterations, time, memory, electrical power, or any combination of these consumed by a computing device when analyzing data. For example, a neural network may more readily identify patterns in data than other approaches. This may enable the neural network to analyze the data using fewer processing cycles and less memory than other approaches, while obtaining a similar or greater level of accuracy. According to embodiments discussed herein, the above-described systems may be utilized to process data and perform modeling operations to generate predictions for target variable, such as a timeframe or a length of stay. These predictions may be used to indicate whether the target variable for real-time or near real-time events occur outside of the predictions and are detected anomalies. For example, systems discussed herein may generate one or more models from patient data and utilize the one or more models to predict the length of stays for events based on the data. The predictions can be used to determine actual or current medical events having length of stays outside of the predicted length of stays within a confidence limit, and identify hospitals having abnormal diagnosis related groups (DRGs) based on the abnormal medical events.

The proposed computerized approach, using systems discussed herein, allows the predictions to reflect a wide variety of patient data and history. This includes patient data retrieved and/or obtained from one or more networked databases over one or more interconnects. The patient data may be claims data, consideration received for medical events, length of stays for the medical events, DRG information for the medical events, and so forth. The DRG information may include information, such as the average (mean) length of stay per DRG, mean length of stay weights, geometric mean, major diagnostic category (MDC), and type. The patient data may further include Medicare data, Medicaid data, centers for Medicare & Medicaid Services (CMS) data, patient demographic data (age and gender), and so forth.

The system allows for an automated analysis of DRG codes and inpatient stays to classify abnormal inpatient stays across a broad range of reasons for inpatient stays while still taking individual patient medical history into account to increase the precision of the modeling. The proposed technique takes a unique approach in that it performs one or more transformations on the patient data including generating one or more subsets of the patient data based on criteria, removing outliers, identify and group patient data based on location, perform a logarithmic transformation, and identify and combine related events. The transformed patient data may further be sampled and used to generate models including the ensemble model. The transformations and sampling reduces computational resource usage, such as processing cycles and memory usage, when generating the models while increasing the precision of the modeling.

Initially, a system may run models based on the transformed and sampled data for a target variable, such as a timeframe or length of stay. The models may be used generate predictions for the target variable that can be used to detect anomalies. The models and predictions may be stored in one or more storage systems including computerized storage devices. After those models have been built and the predictions have been generated, however, computations and processing can be made in a real-time or near real-time fashion as the models and predictions from model runs, which may be retrieved from a storage system, can simply be used to score data or new data sets. This has the potential to allow for a robust, informed approach to flagging lengths of stays associated with events and/or hospitals for review before payment is made for those services.

Moreover, the system allows for the real-time determination of whether a medical claim includes information that is consistent with the predicted length of stays. These real-time determinations cannot occur without the systems discussed herein. For example, a person could not compute the results discussed herein by hand because the amount of information is too much for a person to compute in a reasonable and sufficient amount of time to detect fraud before it occurs. Further, at least a portion of the data used to generate the models and/or make the fraud determinations is gathered from one or more remote databases stored in computerized storage systems connected remotely via one or more computing interconnects. Moreover, the real-time distributed nature of the systems and processing discussed herein solves these large data processing problems.

In one example, systems and techniques discussed herein may include obtaining patient data, the patient data comprising medical events, consideration received for medical events, length of stays for the medical events, and diagnosis related groups (DRGs) for the medical events. The system and techniques also include determining a first subset of the patient data, the first subset including patient data associated with medical events having consideration received in a percentile grouping and determining a second subset of the patient data, the second subset including patient data associated with medical events having consideration received in another percentile grouping.

In some embodiments, the system and techniques include generating a first model based on the first subset of the patient data, the first model for use to determine expected length of stay ranges for each of one or more DRGs, and generating a second model based on the second subset of the patient data, the second model for use to determine the expected length of stay ranges for each of one or more DRGs. Further, the system and techniques also include determining a first quality indication for the first model and a second quality indication for the second model, the first quality indication and the second quality indication based on one or more quality measurements. The first quality indication and the second quality indication may be utilized to select the first model or the second model to score a data set.

In embodiments, the system includes determining the expected length of stay ranges for the DRGs of the patient data using the first model or the second model based on the model indicated as having better quality by the first quality indication and the second quality indication. As previously discussed the model and predictions may be used to detect anomalies, such as medical events outside of the confidence limits (predictions), which may indicate fraud other types of problems, e.g., improper treatments for a diagnosis.

Reference is now made to the drawings, wherein like reference numerals are used to refer to like elements throughout. In the following description, for purposes of explanation, numerous specific details are set forth in order to provide a thorough understanding thereof. It may be evident, however, that the novel embodiments can be practiced without these specific details. In other instances, well known structures and devices are shown in block diagram form in order to facilitate a description thereof. The intention is to cover all modifications, equivalents, and alternatives within the scope of the claims.

Systems depicted in some of the figures may be provided in various configurations. In some embodiments, the systems may be configured as a distributed system where one or more components of the system are distributed across one or more networks in a cloud computing system.

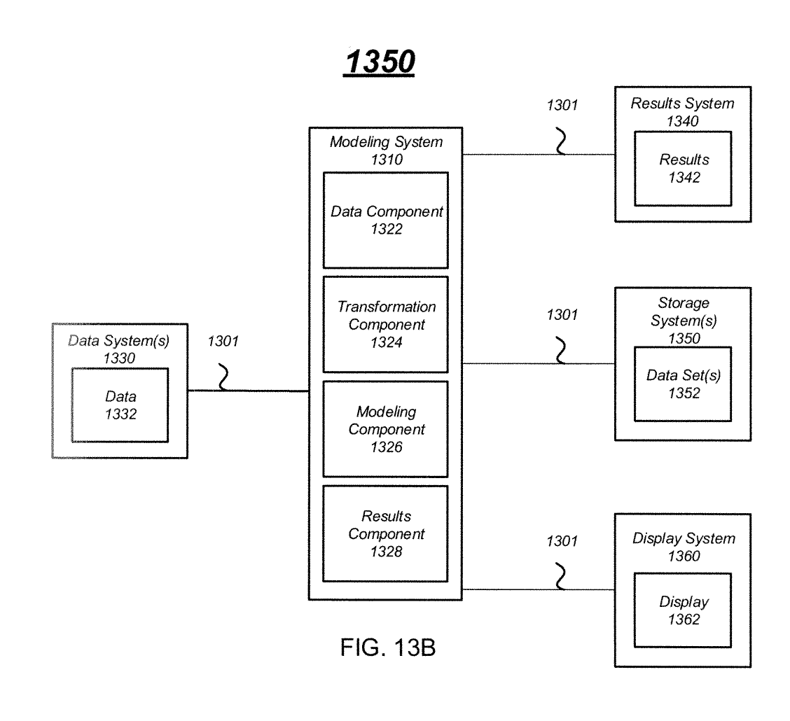

FIGS. 13A/13B illustrate examples of a distributed processing system environment 1300 to process data and determine events that occurred outside of a predicted timeframe based on the data. In embodiments, these operations may be performed in real-time or near real-time by the computing system environment 1300. Further, the illustrated computing system environment 1300 includes a number of systems, components, devices, and so forth to perform these operations; however, embodiments are not limited in the manner In some embodiments, the computing system environment 1300 may include more or less systems, components, and devices, for example.

In some embodiments, the computing system environment 1300 may include a system 1305 having a number of components and is coupled with other systems, including a data system 1330, a results system 1340, and one or more other storage system(s) 1350. Each of the systems 1330, 1340, and 1350 may include a number of networking elements and may be coupled with system 1305 via one or more wired and/or wireless links 1301. Further, the systems 1330, 1340, and 1350 may include any number of storage devices to store information and data, such as data 1332, results 1342, and one or more data sets 1352. The information and data can be stored in any type of data structure, such as databases, lists, arrays, trees, hashes, files, and so forth. Further, the one or more of the systems 1330, 1340, and 1350 can include a Network-attached storage (NAS), Direct-attached storage (DAS), a Storage area network (SAN), include storage devices, such as magnetic storage devices and optical storage devices. The storage may also include volatile and non-volatile storage. Embodiments are not limited in this manner

System 1305 also includes a number components, including, but not limited to, storage 1314, memory 1316, processing circuitry 1318, and one or more interfaces 1320. The system 1305 may be coupled with one or more other systems, components, devices, networks, and so forth through network environment 1335.

Storage 1314 may be any type of storage, including, but not limited to, magnetic storage and optical storage, for example. The storage 1314 may store information and data for system 1305, such as information for processing by the by the system 1305. In embodiments, the storage 1314 may store information, data, one or more instructions, code, and so forth for the modeling system 1310. Embodiments are not limited in this manner.

The memory 1316 of system 1305 can be implemented using any machine-readable or computer-readable media capable of storing data, including both volatile and non-volatile memory. In some embodiments, the machine-readable or computer-readable medium may include a non-transitory medium. The embodiments are not limited in this context. The memory 1316 can store data momentarily, temporarily, or permanently. The memory 1316 stores instructions and data for system 1305, which may be processed by processing circuitry 1318. For example, the memory 1316 may also store temporary variables or other intermediate information while the processing circuitry 1316 is executing instructions. The memory 1316 is not limited to storing the above-discussed data; the memory 1316 may store any type of data.

In embodiments, the system 1305 may include processing circuitry 1318 which may include one or more of any type of computational element, such as but not limited to, a microprocessor, a processor, central processing unit, digital signal processing unit, dual-core processor, mobile device processor, desktop processor, single core processor, a system-on-chip (SoC) device, complex instruction set computing (CISC) microprocessor, a reduced instruction set (RISC) microprocessor, a very long instruction word (VLIW) microprocessor, or any other type of processing circuitry, processor or processing circuit on a single chip or integrated circuit. The processing circuitry 1316 may be connected to and communicate with the other elements of the system 1305 including the modeling system 1310, the storage 1314, the memory 1316, and the one or more interfaces 1320.

The system 1305 may also include one or more interfaces 1320 which may enable the system to communicate over the network environment 1335. In some embodiments, the interfaces 1320 can be a network interface, a universal serial bus interface (USB), a Firewire interface, a Small Computer System Interface (SCSI), a parallel port interface, a serial port interface, or any other device to enable the system 1305 to exchange information.

The system 1305 may also include a modeling system 1310 to generate models to generate predictions for a target variable. The predictions can be utilized to perform real-time analytics to detect anomalies or data outside of the predictions for the target variable. In an example, the modeling system 1310 can generate multiple models that may be combined into an ensemble model to determine predictions for timeframes for a given set of data. The ensemble model is based on multiple models generated based on the data, and the models are evaluated for quality using one or more quality measurements. In one example, the timeframe may be a hospital length of stay for the patients of a large commercial health insurer based on their previous medical events and the location of the events. In another example, the timeframe may be a period of time to perform maintenance on a vehicle based on historical events with similar symptoms.

Embodiments include utilizing the predictions by comparing timeframes for currents events against the predicted timeframes to identify events that are outside of the norm and may require further consideration and inspection. For example, predicted length of stays ranges can be compared to actual length of stays so that investigators may review which patients have significant unforeseen complications, or to understand if certain patients are being discharged from the hospital sooner than would be recommended. In another example, the predicted timeframe may be a period of time for a mechanic to charge to change a transmission based on a given set of symptoms. If the actual time billed is outside the predicted timeframe further investigation may be warranted.

FIG. 13B illustrates an example of the computing system 1350 including further details of the modeling system 1310, which may have a number of components to perform operations discussed herein including generating models to determine predictions for a target variable based on events, the location of events, and other variables. The modeling system 1310 is coupled with one or more data system(s) 1330, results system 1340, and the one or more other storage system(s) 1350 via one or more interconnects 1301. In some instances, the modeling system 1310 may receive and/or retrieve data from one or more of the data system(s) 1330 to generate predictions for the target variable, such as timeframes for events based on the data. The modeling system 1310 may further generate results 1342, as will be discussed in more detail below, and provide the results 1342 to the results system 1340. The results 1342 are based on scoring another data set utilizing the predictions (confidence limits) and the models. The results 1342 include information indicating data of the data set that is outside of the predictions or confidence limits, e.g., 95^{th} percentile.

In embodiments, the modeling system 1310 may include a data component 1322, a transformation component 1324, a modeling component 1326, and a results component 1328 to process data, generate models and an ensemble model, generate predictions for a target variable, and generate results 1342 based on the predictions. The modeling system 1310 may further be coupled to a display system 1360 having a display device 1362 via one or more interconnects 1301. The modeling system 1310 can present the results 1342 and data identified as outside of the predictions to a user in a presentation on a display device 1362. For example, the results 1342 may be presented in a graphical user interface (GUI) to enable a user to determine predicted timeframes for events and easily detect events having timeframes outside of the predicted timeframes.

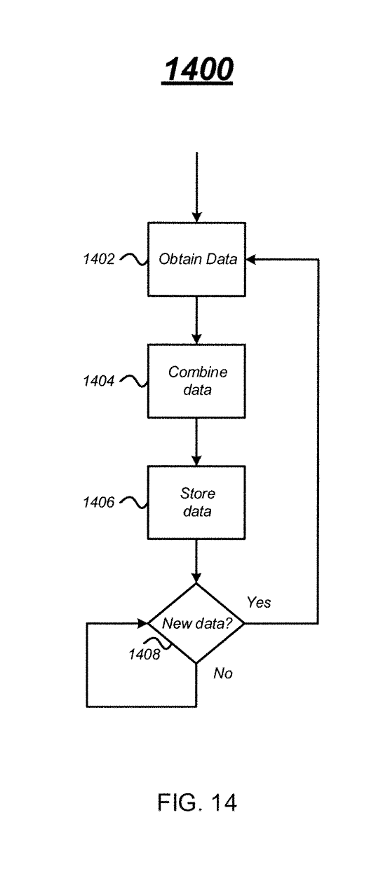

In embodiments, the modeling system 1310 including the data component 1322 may collect and/or receive data from various sources, group the data into a data set, and make the data set available for other components of the modeling system 1310 to use in generating predictions for the target variable, e.g. predicted timeframes for events. FIG. 14 illustrates one possible logic flow 1400 that may occur during operation of a data collection routine performed by the data component 1322 to generate a data set. At block 1402, the data component 1322 may obtain data from one or more sources, such as data system 1330, which includes one or more databases, network entities, websites, data servers, and so forth. The data may be retrieved or received from a number of databases, each having different parts of the data, for example. In one specific example, the data may be patient data including claims data, consideration received for medical events, length of stays for the medical events, diagnosis related groups (DRG) information for the medical events, and so forth. The DRG is a patient classification scheme which provides a means of relating the type of patients a hospital treats to the costs incurred by the hospital. More specifically, the DRG is a statistical system that is used to classify any inpatient stay into groups for the purposes of payment. The DRGs are divided into twenty major body systems and subdivided into 467 groups. The DRG information may include information, such as the average (mean) length of stay per DRG, mean length of stay weights, geometric mean, major diagnostic category (MDC), and type. The patient data may further include Medicare data, Medicaid data, centers for Medicare & Medicaid Services (CMS) data, patient demographic data (age and gender), and so forth. In this example, the claims data may be obtained from one or more databases owned and/or operated by an insurance company, while the CMS data may be obtained from one or more databases owned and/or operated by CMS. The other patient data may come from different sources and embodiments are not limited in this manner

In another example, the data obtained may be vehicle data including vehicle insurance claims data (bills), vehicle diagnostic data, repair data (average hours billed), EBSCO information services vehicle data, American Automobile Association (AAA) data, and so forth. The vehicle data may also be obtained from various sources, e.g., databases owned/operated by EBSCO vehicle services, databases owned/operated by AAA, insurance company's databases, and so forth. Embodiments are not limited in this manner.

In embodiments, at block 1404, the logic flow 1400 includes combining the data into a data set that may be used by other components of the modeling system 1310, for example. In some embodiments, the data component 1322 may perform an initial evaluation of the data to filter out data that has quality issues, e.g., missing information, fields, data, etc. The data component 1322 may use the remaining data and combine the data into a data set and store the data set in storage at block 1406. For example, the data component 1322 may store the combined data as data set 1352 in storage system 1350. The data set 1352 may then be retrieved from the storage system 1350 and used by other components of the modeling system 1310.

The logic flow 1400 may also include checking whether new data is available at block 1408. For example, the data component 1322 may check whether new data is available from one or more of the sources on a periodic or semi-periodic basis. In other instances, the data component 1322 may receive an indication from a source that new data is available. Embodiments are not limited in this manner. If new data is detected, the logic flow 1400 may repeat itself any number of times and/or until the modeling system 1310 is complete in generating one or more timeframes for events.

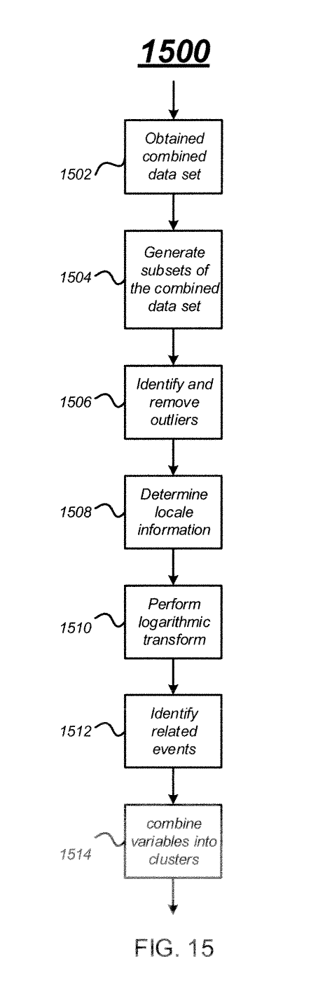

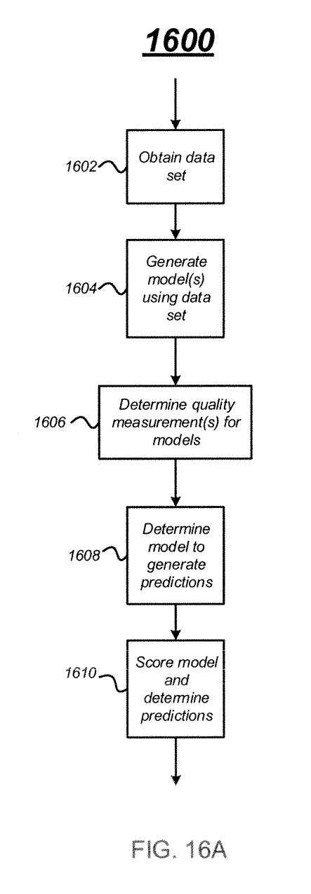

With reference to FIG. 13B, the modeling system 1310 may include a transformation component 1324 to perform one or more transformations on a data set, such that one or more models may be generated that may be used to determine predicted timeframes for events. These transformations may include grouping the data of a data set into one or more percentile groups, e.g. flag the top twenty-fifth percentile (25%) of costs of a patient's medical claims associated with a particular service or diagnosis, flag the top seventy-fifth percentile (75%) of costs of a patient's medical claims associated with a particular service or diagnosis, etc., removing data not within at least one of these percentile groups, removing outliers from the data, group and/or flag data based on location, identify related events, combine the data into subsets based on selected variables, and so forth. FIG. 15 illustrates one possible logic flow 1500 for performing one or more transformations on a data set for use in generating model(s) to generate predictions for a target variable, e.g., timeframes for events by components of the modeling system 1310. Reference is made to FIG. 15.

At block 1502, the logic flow 1500 includes obtaining a data set from storage, such as data set 1352 from storage system 1350. The data set may include a combination of data collected from one or more sources of information. In one example, the data set may be patient data related to hospital patients and lengths of stays. In another example, the data set may be vehicle data related to vehicle maintenance and a timeframe to fix a problem. Embodiments are not limited in this manner, and a data set may include data relating other topics, such as predicting system downtime based on computer failures, a timeframe to fix other vehicles (airplanes, trains, subways, etc.), and so forth.

In embodiments, the logic flow 1500 includes determining one or more subsets of the data set based on one or more criteria at block 1504. The data set may be broken into the different subsets to illustrate a severity of a problem, for example, and the one or more criteria may be used to determine the level of severity of the problem. For example, the transformation component may determine a subset of patient data associated with patient's having consideration received or amounts paid for medical events in a percentile group, e.g., the top 25 percent. Data grouped into the top 25 percentile subset may be associated with patients having a severe case of a diagnosis because it groups the highest paying patients. The transformation component may determine another subset of patient data associated with patient's having consideration received or amounts paid for medical events in another percentile group, e.g., the top 75 percent. Data grouped into the top 75 percentile subset may be associated with patients having a disease associated with a diagnosis. The bottom 25 percent may be filtered out of the data to remove accidental, incorrect, and spurious information, e.g., incorrect diagnoses code used for claims. In this example, the medical events include payment for a diagnosis, payment for utilizing an emergency room, a number of office visits, particular claim codes, and so forth. Further, each of the percentile groups may or may not have overlapping data. Embodiments are not limited to these examples. For example, the percentiles utilized may be different and may be chosen to adequately group data for a desired severity level. Further, additional percentile groups may be determined based on different criteria and/or other percentiles may be utilized to generate the groups, for example.

The subsets may be identified in the data set using flags or other indicators. For example, the data set may be stored in a database and a column of the database may indicate whether an entry is a member of the first subset or not and another column of the database may indicate whether an entry is a member of the second subset or not. In one specific example, a first column may indicate whether entries of the data set are members of the top 25 percentile and a second column may indicate whether entries of the data set are members of the top 75 percentile. Embodiments are not limited in this manner.

In some embodiments, the logic flow 1500 includes identifying and removing outliers from the data set at block 1506. In one example, the outliers may be determined for predicted timeframes as well as a confidence interval. Data associated with timeframes outside the confidence threshold may be removed from the data set and subsets prior to generating the models. In a specific example, the predicted length of stay may be calculated as well as a confidence interval. An outlier may be actual length of stay that is outside a confidence threshold (a particular confidence interval, such as 95% or 6+standard deviations from the Winsorized mean). Thus, data associated with the identified outlier may be removed, e.g., not used when generating the models, from the data set and subsets. The confidence threshold is configurable and can be higher or lower based on a user or computer configuration. Embodiments are not limited in this manner.

The logic flow 1500 includes determining locale information for the data set and subsets at block 1508. More specifically, each event may be associated with a location where the event took place. The location may be identified as a city, a borough, a township, a county, a region within a state, a state, a region of the country, by country, and so forth. The locale information may be used as an input when generating the models. For example, in the healthcare example, the country of a facility where the inpatient stay occurred may be identified because care choices can vary greatly based on the location where the care occurred. This locale information becomes an input to the model(s) to adjust for the location where the care occurred. In another example, a region of a country may be identified in the automotive example due to varied prices and timeframes to fix a vehicle based on location. Embodiments are not limited to these examples.

At block 1510, the logic flow 1500 includes performing one or more logarithmic transformations on the data set. For example, the target variable, e.g., a predicted timeframe, may be converted into Log10. Other variables of the data set may also be converted into Log10. For example, variables that are determined to have a long tail distribution and are far away enough from a normal distribution may be converted to Log10. Embodiments are not limited in this manner.

In embodiments, the logic flow 1500 includes identifying related events for each of the events in the data set at block 1512. For example, multiple events may be related to the problem or symptoms, e.g., the same illness or medical event may require multiple admissions to the hospital. For example, embodiments may include identifying claims associated with a readmission within a period of time of a date of a current admission for each of the claims for use as a variable in generating the models. In another example, the same problem with a vehicle may be require multiple trips to the auto mechanic These readmissions and multiple trips may be identified and flagged in the data set. In some instances, two or more events may not be considered related if they do not occur within a specific timeframe. For example, only inpatient stays that occurred within 30 days before the admission of a first stay may be identified and flagged as related. The related events may be flagged and may be used to indicate that the second event is related to the first event and may be a complication of the first event. The related events may be flagged to indicate the payment received for the second event may be affected, e.g., lower than a payment received if the event was not a readmission. In some instances, a facility may not get payment from a healthcare provider for a readmission. Similarly, a car mechanic may charge less for follow-up repairs related to a problem that was not fixed the first time. Embodiments are not limited to these examples.

The logic flow 1500 also includes combining variables into pre-defined clusters at block 1514. Combining variables reduces the size of the data set and subsets, which saves on resource utilization, e.g., processing cycles and memory usage. In embodiments, dimension reduction procedure is performed to determine variables that are highly correlated. Variables may be determined correlated utilizing a correlation technique, such as Pearson, Kendall, or Spearman. A large number of variables can be replaced with a fewer correlated variables with little loss of information. Variables may be considered highly correlated when a correlation value is above a correlation threshold, which may be user or computer set. Embodiments are not limited in this manner.

In some embodiments, the data set and the one or more subsets may be utilized to generate one or more models after one or more transformations are performed, as discussed above with respect to FIG. 15. The transformed data set and subsets may be stored in storage 1350 as data set(s) 1352 by the transformation component 1324 and may be used as training set(s) to generate the one or more models, for example. In some embodiments, the data set 1352 including the subsets, which may be identified by flags, may be sampled to generate the training sets. In some embodiments, the sampling may be based on a number of members in the data set and ensure that there is no overlap, e.g., that they are mutually exclusive. Once sampled, the transformation component 1324 may identify events of the same type having a count below a minimum threshold, which may be user or computer set. The minimum threshold can be based on a minimum number of counts to have a sample that can be statistically modeled. The minimum threshold is statically based depending on a number of input variables. The transformation component 1324 may add events (and associated data) back into the training set(s) that are identified are having a count below the minimum threshold to ensure a sample can be statistically modeled. These events may be added back at random.

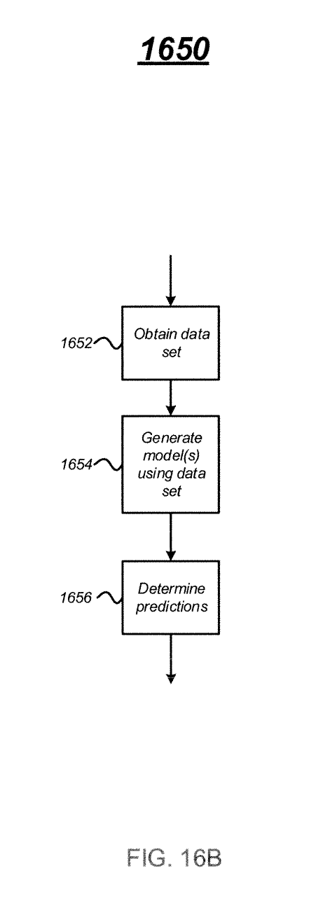

With reference, to FIG. 13B, the modeling component 1326 may utilize the training set(s) to generate one or more models that may be used to generate predictions for the target variable based on the events. The predictions may be used to process other data sets to identify data that is not within the predicted results for the target data. The generated models may further be used to generate an ensemble model and predictions for a targeted variable, as will be discussed in more detail below. FIG. 16A/16B illustrate possible logic flows 1600 and 1650 to process data, generate one or more models based on the data, and generate predictions for a target variable by the modeling component 1326. FIG. 16A illustrates one possible logic flow 1600 utilizing quality measurements to select a model, and using the selected model to generate to score a data set. Reference is now made to FIG. 16A. At block 1602, the logic flow 1600 includes obtaining a data set for use to generate one or more models. The data set may be obtained from storage, such as storage system 1350, for example. The data set includes data after one or more transformations are performed, as discussed above with respect to FIG. 15. Further, the data set may include a sampling of a larger data set.

At block 1604, the logic flow 1600 includes generating one or more models using the data set. In embodiments, the one or more models may each be a generalized linear mixed model in which the linear predictor contains random effects and fixed effects. One or more operations may be performed to fit generalized linear mixed models based on linearization using the data set with random effects. In one example, the generalized linear mixed models may be generated by using PROC GLIMMIX by SAS Institute Inc. In one example, a first model may be generated utilizing a sampling of the subset of data including the top 25 percentile grouping of amounts paid, and a second model may be generating utilizing a sampling of the subset of data including the top 75 percentile grouping of amounts paid. As previously discussed, the bottom 25 percent based on amounts paid may be filtered out to remove possible erroneous information. However, in some embodiments, a third model may be generated utilizing a sampling of the entire data set including the bottom 25 percent. Embodiments are not limited to these examples. As previously mentioned different subsets may be generated using different percentile groupings and used to generate one or more models, for example.

At block 1606, one or more quality measurements may be determined for each of the models. For example, a maximum likelihood estimation method based on integral approximation may be utilized and a quality score may be generated for each models. The maximum likelihood estimation method may output an Akaike Information Criterion-Correct (AICc) estimation, e.g., a quality score, for each of the models which may be a relative estimation of quality for a given set of data. More specifically, the quality score for each model indicates a relative quality score compared to the other models for the data set. The model with the lowest AICc estimation score has the best quality compared to the other models having higher scores.

In embodiments, additional quality measurements may be outputted when generating the models. For example, parameter estimations for each of the models may be determined. In one example, the parameter estimations may be used to determine which events are significant, e.g., PROBT<ALPHA (0.05-95% confidence level). Embodiments also include determining other information from the models including observations with “perfect predictions,” e.g., is the predicted timeframe within the actual timeframe, for each of the models. The AICc estimation of the models, output parameter estimates, and predictions matching actual timeframes may be quality measurements or quality indications used to select a model to score. At block 1608, the logic flow includes selecting a model to score a data set based on the one or more quality measurements. For example, the model having the most significant DRGs, higher counts of perfect predictions, and lower AICc may be chosen to score the entire data to generate predictions.

At block 1610, each of the predictions for the target variable for each of the events may be determined from the selected model by scoring the entire data set. For example, the upper and lower confidence limits, for each of the events is determined. The upper and lower confidence limits may be the lower bound of a predicted length of stay and the upper bound of the predicted length of stay. The ranges may be specific for each event. For example, the range may be specific by DRG for each patient and predicted ranges are not the same on DRGs across patients, e.g., a patient may have a predicted range of 2-6 days for a particular DRG, but another patient could have a different predicted range for the same particular DRG. The variability of the lengths of stay for a particular DRG is a due to a number of reasons such as the history of a particular patient, and the patient's previous use of the medical system.

FIG. 16B illustrates another possible logic flow 1650 utilizing an ensemble model and voting to determine predictions or confidence limits for the target variable. Reference is now made to FIG. 16B. At block 1652, logic flow 1650 includes obtaining a data set for use to generate one or more models. As discussed, the data set may be obtained from storage, such as storage system 1350, for example. The data set includes data after one or more transformations are performed, as discussed above with respect to FIG. 15. Further, the data set may include a sampling of a larger data set and subsets.

At block 1654, the logic flow 1650 includes generating one or more models using the data set. In embodiments, the one or more models may each be a generalized linear mixed model in which the linear predictor contains random effects and fixed effects, as similarly discussed above. In one example, a first model may be generated utilizing a sampling of the subset of data including the top 25 percentile grouping of amounts paid, and a second model may be generating utilizing a sampling of the subset of data including the top 75 percentile grouping of amounts paid. As previously discussed, the bottom 25 percent based on amounts paid may be filtered out to remove possible erroneous information. However, in some embodiments, a third model may be generated utilizing a sampling of the entire data set including the bottom 25 percent. Embodiments are not limited to these examples. As previously mentioned different subsets may be generated using different percentile groupings and used to generate one or more models, for example.

At block 1656, the data set may be scored using each of the models generated at block 1654 to determine the most appropriate confidence limits. The data set is scored with all three models, e.g. top 25 percentile grouping, top 75 percentile grouping, and full data set, for example. A lower confidence limit and upper confidence limit are determined for the target variable for all three models. Voting is applied to determine which lower confidence limit and upper confidence limit. For example, if two or more of the models agree on the same lower confidence limit, that value is used for the lower confidence limit. If all three models choose a different confidence limit, the middle value is used for the lower confidence limit. The same approach is applied to determine the upper confidence limit. For example, if three or two of the models agree on the same upper confidence limit that value is used as the upper confidence limit. However, if none of the models agree on the upper confidence limit, the middle value is chosen. The defined confidence limit may be based on a number of outliers wanted for a giving set of predictions and the volume of the data set used to transform, sample, and generate the models. In one example, the defined confidence limited may be the 95^{th} percentile. Embodiments are not limited in this manner and the defined confidence limit may be adjusted by a user or the system based on a desired number of outliers and/or the volume of the data set.

As discussed, the upper and lower confidence limits, for each of the events may be timeframe or predicted length of stay for a diagnosis. In one specific example, minimum and maximum predictions by DRG by patient are combined to create a range of predicted length of stay (e.g., “2(min)-6(max) days”). Note that ranges may be specific for each event. For example, the range may be specific by DRG for each patient and predicted ranges are not the same on DRGs across patients, e.g., a patient may have a predicted range of 2-6 days for a particular DRG, but another patient could have a different predicted range for the same particular DRG. The variability of the lengths of stay for a particular DRG is a due to a number of reasons such as the history of a particular patient, and the patient's previous use of the medical system. Embodiments are not limited to this example.

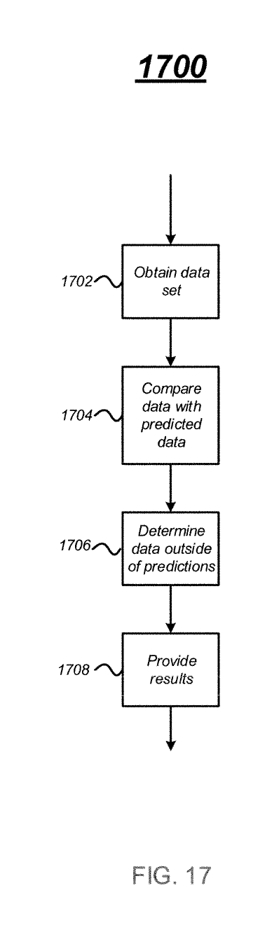

With reference to FIG. 13B, the results component 1328 can apply the selected model when quality measurements are utilized or the ensemble model to score other data sets not used to for training. The predictions may be used to detect data outside of the predicted ranges, for example. This information can be used to identify abnormalities, e.g., events whose length of stay are outside the confidence limits predicted for the length of stay, and may indicate that further inspection is required. FIG. 17 illustrates one possible logic flow 1700 to apply the predictions to other data sets to detect abnormal events by the results component 1328.

At block 1702, the logic flow 1700 includes obtaining a data set to score based on the predictions generated from training. In embodiments, the data set may be obtained from one or more sources, such as an insurance company, a hospital, one or more public and/or private databases, and so forth. At block 1704, embodiments include scoring the data set including comparing the data in the data set with the predictions. For example, embodiments include comparing actual length of stays with predicted length of stays. In another example, embodiments may include comparing an actual timeframe to fix a vehicle to a predicted timeframe. Further and at block 1706, embodiments may include determining data outside of the predicted ranges, e.g., actual length of stays outside of the predicted length of stays or actual maintenance timeframes outside of predicted timeframes. The data associated with the identified abnormal variable may be flagged for further inspection. At block 1708, the results of the analysis of the data set may be provided to a system that can further inspect the detected anomalies, such as results system 1340 as results 1342. In some embodiments, the results may be displayed on a display in a graphical user interface (GUI). For example, data associated with the identified abnormal variable may present or highlighted in the GUI. In some instances, results system 1342 may further process the data to detect patterns and highlight particular locales having anomalies greater than a specified threshold, e.g., 10% more anomalies than other locales. The results system 1342 may highlight these areas on a map in the GUI, example. In another example, the results system 1342 may narrow the anomalies down to a particular hospital and highlight the particular hospital on a map in the GUI. Embodiments are not limited to these examples.

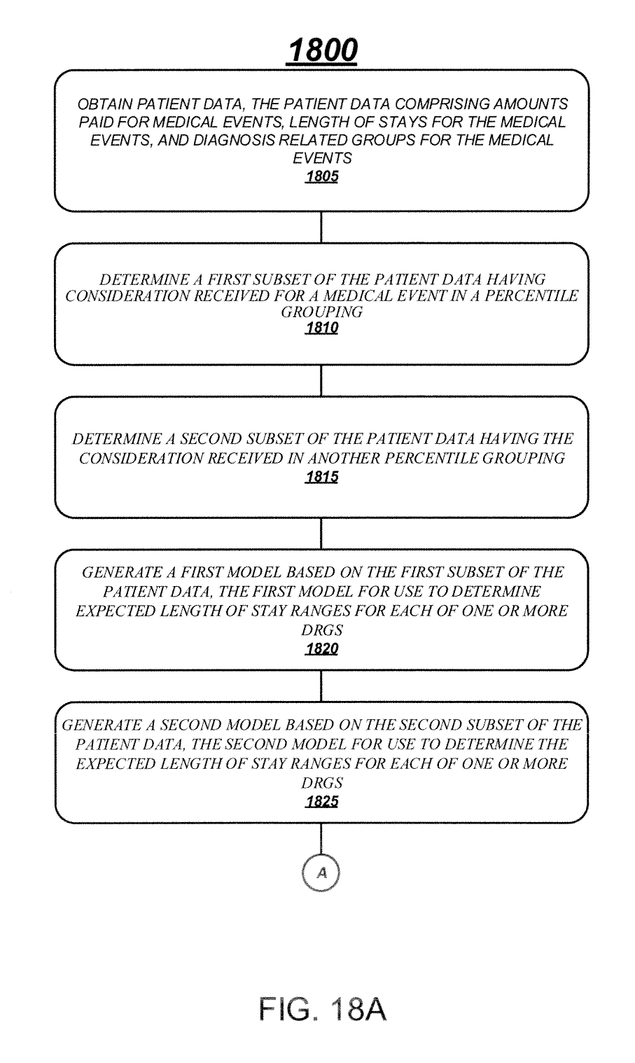

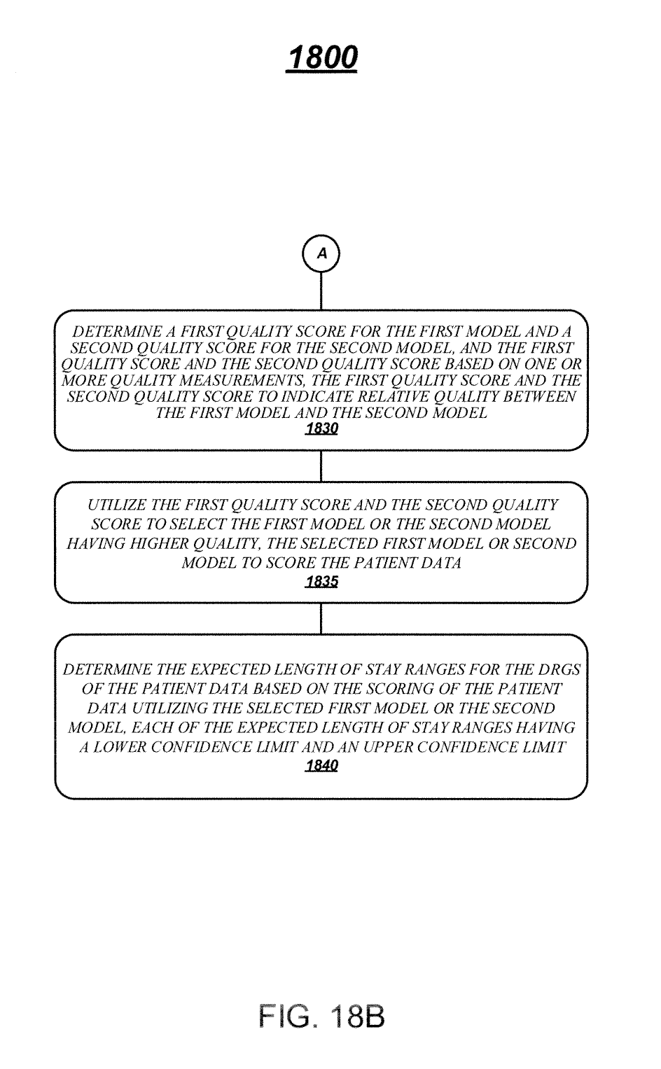

FIGS. 18A/18B illustrate an example of a logic flow diagram 1800. The logic flow 1800 may be representative of some or all of the operations executed by one or more embodiments described herein. For example, the logic flow 1800 may illustrate operations performed by the modeling system 1310, as discussed in Figures FIGS. 13A-17, and FIGS. 19A-19E. In the illustrated embodiment shown in FIGS. 18A/18B, the logic flow 1800 may include obtaining patient data, the patient data comprising medical events, consideration received for medical events, length of stays for the medical events, and diagnosis related groups (DRGs) for the medical events at block 1805. The patient data may be obtained from one or more sources which include one or more databases, network entities, websites, data servers, and so forth. The data may be retrieved or received from a number of databases, each having different parts of the data, for example, and may be coupled via one or more network interconnects. The patient data from the one or more sources may be combined to generate a data set on which one or more transformations may be performed.

In embodiments, the logic flow 1800 includes determining a first subset of the patient data having consideration received for a medical event in a percentile grouping at block 1810. For example, the first subset may be the top 25% of consideration received or amounts paid for a medical event, such as a diagnosis or an emergency room visit, compared to other amounts paid for the same medical event. Further and at block 1815 the logic flow 1800 includes determine a second subset of the patient data having the consideration received in another percentile grouping. The second subset may be the top 75% of consideration received or amounts paid for a medical event compared to other amounts paid for the same medical event. Embodiments are not limited to these examples. Embodiments may include more or fewer percentile groups and/or utilize different percentile thresholds.

At block 1820, the logic flow 1800 includes generating a first model based on the first subset of the patient data, the first model for use to determine expected length of stay ranges for each of one or more DRGs. At block 1825, the logic flow 1800 includes generating a second model based on the second subset of the patient data, the second model for use to determine the expected length of stay ranges for each of one or more DRGs. Note that in some instances, one or more additional transformations may be applied to the patient data including the first subset and the second subset. For example, outlying data may be identified and removed, locale information may be indicated, one or more logarithmic transformations may be applied, related events may be identified and flagged, and correlated variables may be combined into clusters. Further, the patient data and subsets may also be sampled and the samples may be used training data to generate the models.

At block 1830, the logic flow 1800 includes determining a first quality indication for the first model and a second quality indication for the second model, and the first quality indication and the second quality indication based on one or more quality measurements, the first quality indication and the second quality indication to indicate relative quality between the first model and the second model. The first and second quality indications may be based on one or more quality measurements, such as AICc measurement for the models, output parameter estimates, and identifying a number of predictions matching actual timeframes. In one example, the first or second model having the lowest AICc measurements may be identified as having the comparatively best quality of the first or second model. In another example, the first or second model having the highest number “perfect predictions,” maybe identified as having the comparatively best quality. In some instances, a combination of the quality indications may be used to select a model. For example, the model with the most significant DRGs, with higher counts of perfect predictions, and a lower AICc will be used for predictions. Embodiments are not limited in this manner.

At block 1835, the logic flow 1800 includes utilizing the first quality indication and the second quality indication to select the first model or the second model having higher quality, the selected first model or second model used to score the patient data. As mentioned, the model having a better quality indication is selected. The logic flow 1800 includes determine the expected length of stay ranges for the DRGs of the patient data based on the scoring of the patient data utilizing the selected first model or the second model, each of the expected length of stay ranges having a lower confidence limit and an upper confidence limit at block 1840. The predictions of the expected length of stay ranges may be used to compare to other data sets to detect abnormal data, e.g., data not within the predicted ranges.

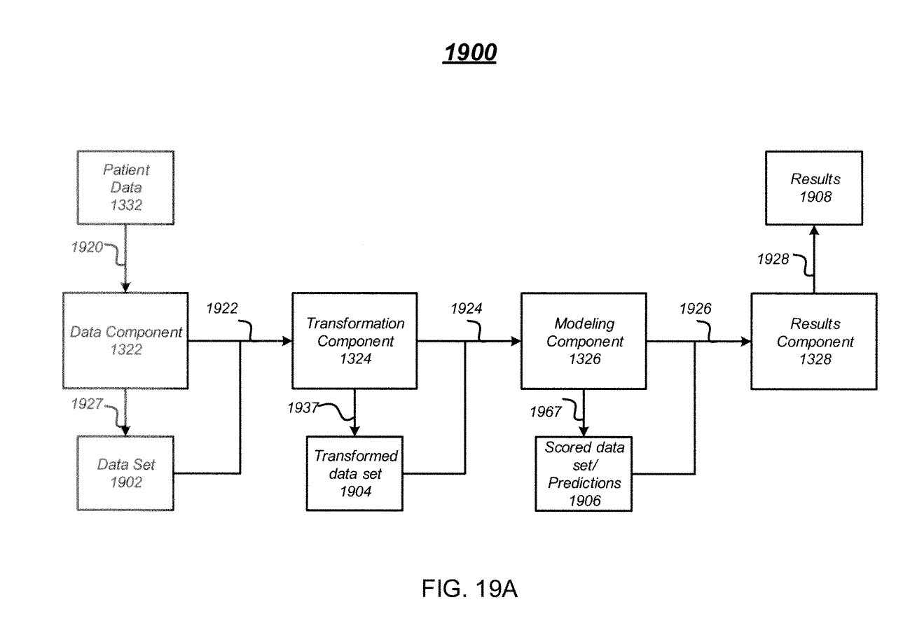

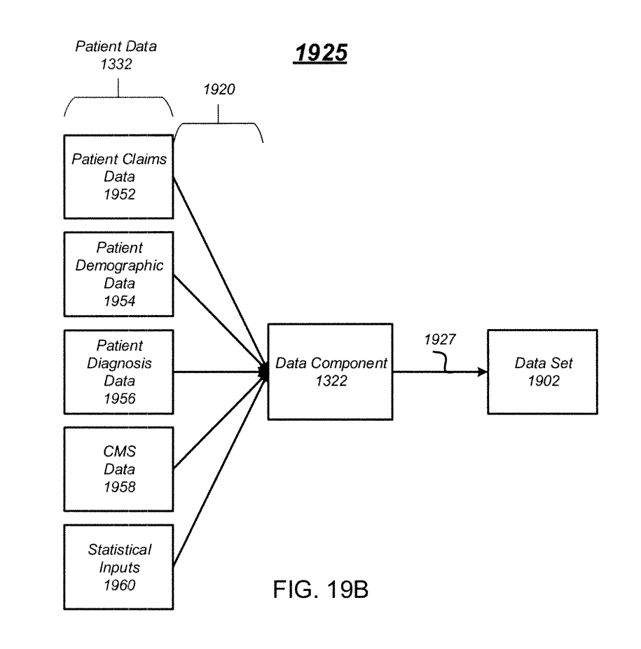

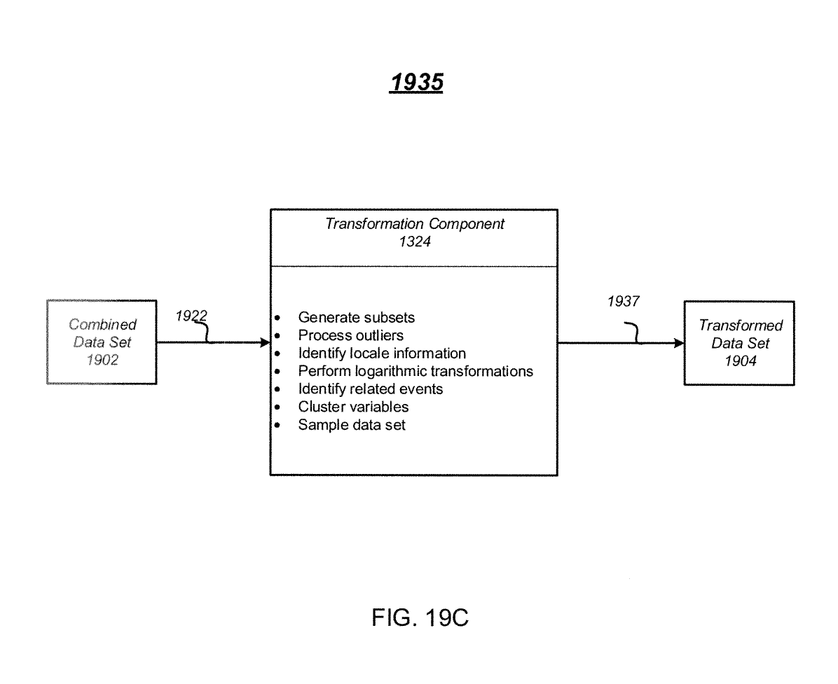

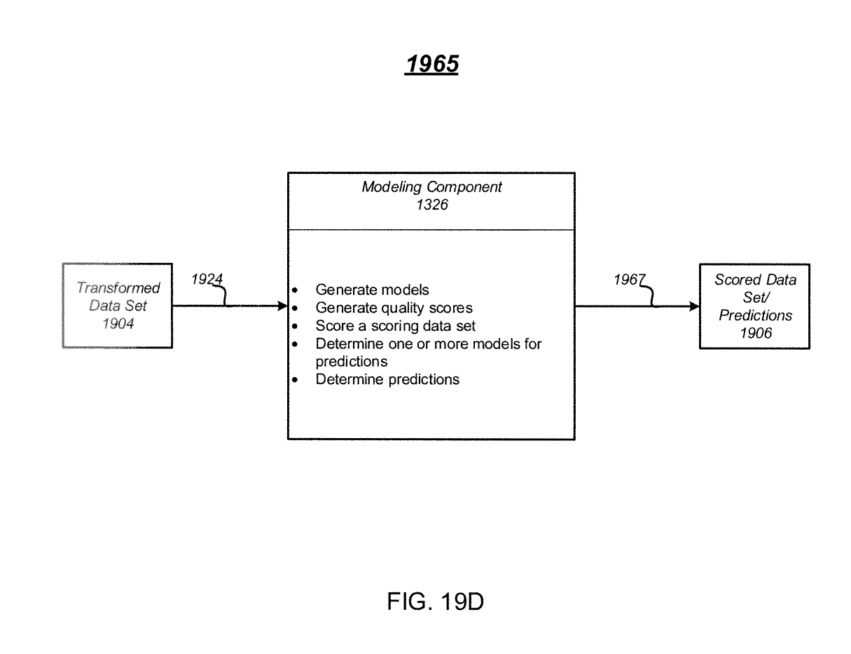

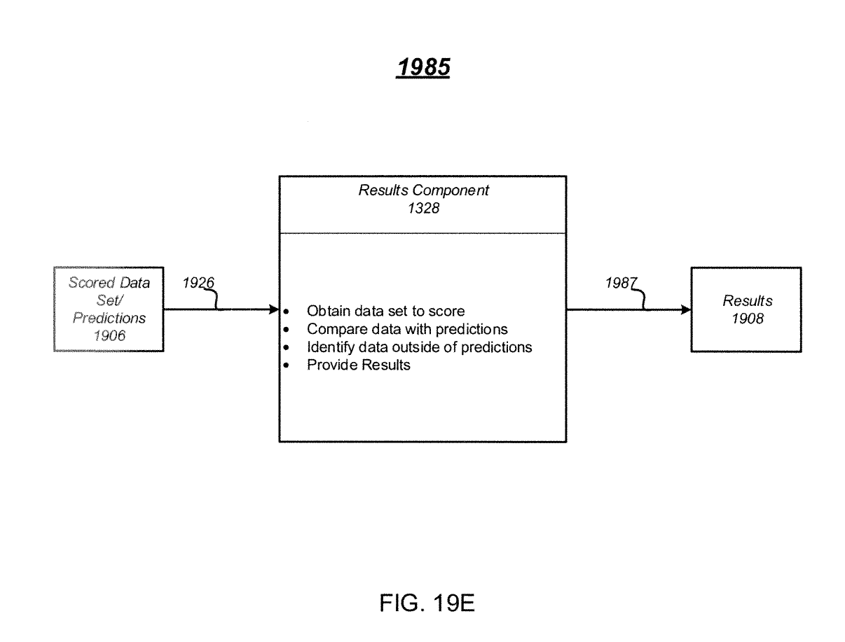

FIGS. 19A-19E illustrate system processing flows to perform training utilizing a data set to generate predictions for a target variable. The illustrated example includes processing patient data 1332 to generate predictions for length of stays for DRGs per patient. However, embodiments are not limited to the example, and as previously discussed can be applied to other concepts to generate predictions for a target variable based on a data set and training. FIG. 19A illustrates an overview system processing flow 1900 to perform training and generate predictions, while FIGS. 19B-19E illustrate more detailed processing flows 1925, 1935, 1965, and 1985, respectively, of one or more operations for system processing flow 1900. These and other details will become more apparent in the flowing description.

In the illustrated system processing flow 1900, a data component 1322 may obtain patient data 1332 at line 1920. The patient data 1332 may be obtained data from one or more sources, such as one or more databases, network entities, websites, data servers, and so forth. FIG. 19B illustrates a detailed processing flow 1925 of the data component 1322 obtaining patient data 1332. In the illustrated example, the patient data 1332 includes patient claims data 1952, patient demographic data 1954, patient diagnosis data 1956, CMS data 1958, and statistical inputs 1960. The patient claims data 1952 may further include claims data, amounts paid or consideration received for medical events, length of stays for the medical events, DRG information for the medical events, and so forth. The patient demographic data 1954 includes information such as the location of a patient, age of a patient, gender of a patient, height of a patient, the weight of a patient, and so forth. The patient diagnosis data 1956 includes information, such as the average (mean) length of stay per DRG, mean length of stay weights, geometric mean, major diagnostic category (MDC), and type. The CMS data 1958 includes information such as Medicare data, Medicaid data, US DRG averages for Medicare and Medicaid, and so forth.

The data component 1322 may obtain the data and process the data including generating a data set 1902 using the obtained data at line 1920. Further, the data component 1322 may perform an initial scan of the data to filter out data that has quality issues, e.g., missing information, fields, data, DRGs with quality issues (with four digits), etc. The data component 1322 may use the remaining data and combine the data into the data set 1902 and store the data set in storage on a computer storage device at line 1927. For example, the data component 1322 may store the combined data as data set 1352 in storage system 1350. The data set 1902 may then be retrieved from the storage system 1350 and used by other components of the modeling system 1310.

In one example, the transformation component 1324 may obtain the data 1902 at line 1922 as illustrated in FIG. 19A. The transformation component 1324 may perform one or more transformations on the data set as illustrated in FIG. 19C. For example, the transformation component 1324 may generate one or more subsets of the data set. The generating one or more subsets of the data set may be based on one or more criteria. For example, the data set may be broken into percentile groups. The percentile groups for a data set including patient data 1332 may be generated based on consideration received for each diagnosis in the data set. For example, the transformation component may determine a subset of patient data associated with patient's having amounts paid for medical events in a percentile group, e.g., the top 25%. The transformation component may determine another subset of patient data associated with patient's having amounts paid for medical events in another percentile group, e.g., the top 75%. These subsets may be used to generate models.

In embodiments, the transformation component 1324 may perform additional transformations, such as processing outliers. For example, the transformation component 1324 may identify and remove outliers from the data set. In one example, the outliers may be determined for predictions made for the target variable, e.g., lengths of stays, as well as a confidence interval. Data associated with lengths of stays outside of a confidence threshold may be removed from the data set including the subsets prior to generating the models. The predicted length of stay may be calculated per DRG as well as a confidence interval. An outlier is any actual length of stay that is outside a confidence threshold, e.g., a particular confidence interval, such as 95% or 6+standard deviations from the Winsorized mean. Thus, data associated with the identified outlier may be removed from the data set and subsets.

The transformation component 1324 may also identify locale information for the data set. More specifically, each event may be associated with a location where the event took place. The locale information may be used as an input when generating the models. For example, the county of a facility where the inpatient stay occurred may be identified because care choices can vary greatly based on the location where the care occurred. This locale information becomes an input to the models to adjust for the location where the care occurred.

In embodiments, the transformation component 1324 may perform one or more logarithmic transformations on the data set. For example, the target variable, e.g., a predicted length of stay, may be converted into Log10. Other variables of the data set may also be converted into Log10. For example, variables that are determined to have a long tail on the distribution and are far away enough from a normal distribution may be converted to Log10. Embodiments are not limited in this manner.

The transformation component 1324 may also identify related events for each of the events in the data set. For example, multiple events may be related to the problem or symptoms, e.g., the same illness or medical event may require multiple admissions to the hospital. For example, embodiments may include identifying claims associated with a readmission within a period of time of a date of a current admission for each of the claims for use as a variable in generating the models. In another example, the same problem with a vehicle may require multiple trips to the auto mechanic These readmissions and multiple trips may be identified and flagged in the data set. In some instances, two or more events may not be considered related if they do not occur within a specific timeframe. For example, only inpatient stays that occurred within 30 days before the admission of a first stay may be identified and flagged as related. The related events may be flagged and may be used to indicate that the second event is related to the first event and may be a complication of the first event. The related events may be flagged to indicate the payment received for the second event may be affected, e.g., lower than a payment received if the event was not a readmission. In some instances, a facility may not get payment from a healthcare provider for a readmission. Similarly, a car mechanic may charge less for follow-up repairs related to a problem that was not fixed the first time. Embodiments are not limited to these examples. The related events may be flagged by putting an indication as a database entry indicating that the event is related to another event. The flag or indication may indicate the other related event. The flag may be used during model as parameter to indicate the payment associated with the event may be affected. Embodiments are not limited to this example.

In embodiments, the transformation component 1324 may cluster one or more variables. For example, the transformation component 1324 may combine variables into pre-defined clusters. Combining variables reduces the size of the data set and subsets, which saves on resource utilization, e.g., processing cycles and memory usage. More specifically, a dimension reduction procedure is performed to determine variables that are highly correlated. Variables may be determined correlated utilizing a correlation technique, such as Pearson, Kendall, or Spearman. Embodiments are not limited to these examples. For example, a large number of variables can be replaced with a few with little loss of information. Variables may be considered highly correlated when a correlation value is above a correlation threshold, which may be user or computer set. Embodiments are not limited in this manner

In embodiments, the transformation component 1324 may also sample the data set to generate the transformed data set 1904, e.g., a training set. In some embodiments, the sampling may be based on a number of members, e.g., patients, in the data set and ensure that there is no overlap, e.g., that they are mutually exclusive. Once sampled, the transformation component 1324 may identify events of the same type having a count below a minimum threshold, which may be user or computer set. The transformation component 1324 may add events (and associated data) back into the training set that is identified are having a count below the minimum threshold. In embodiments, the transformation component 1324 may store the transformed data set 1904 in storage for use by other components, such as the modeling component 1326. The transformed data set 1904 including the subsets may be utilized to generate one or more models.

In FIG. 19A, the modeling component 1326 may obtain the transformed data set 1904 at line 1924. The transformed data set 1904 may include one or more subsets of data identified by one or more respective flags, e.g., top 25% flag, top 75% flag, and so forth. The modeling component 1326 may obtain the transformed data set 1904 and perform one or more operations as illustrated in more detail in FIG. 19D.

Embodiments include the modeling component 1326 generating one or more models using the transformed data set 1904 including one or more subsets. For example, the modeling component 1326 may generate a first model for a first subset, e.g., the top 25 percent group with data associated with the top 25% flag, and a generate a second model for a second subset, e.g., the top 75 percent group with data associated with the top 75% flag. In some embodiments, a third model utilizing the entire transformed data set may be generated. The one or more models may each be a generalized linear mixed model in which the linear predictor contains random effects and fixed effects. One or more operations may be performed to fit generalized linear mixed models based on linearization using the data set with random effects. In one example, the generalized linear mixed models may be generated by using PROC GLIMMIX by the SAS Institute Inc.

The modeling component 1326 may then score one or more models and generate predictions. The modeling component 1326 may utilize different approaches to determine one or more of the models to generated the predictions, as previously discussed. In one example, the modeling component 1326 may determine quality indications for each of the models and select a model to score based on the quality indications. The quality indications are based on one or more quality measurements and criteria. For example, a maximum likelihood estimation method based on integral approximation may be utilized and a quality indication may be generated for each model. The maximum likelihood estimation method may output an AICc estimation for each of the models which may be a relative estimation of quality for a given set of data, e.g., a subset, or the entire transformed data set. In one example, the quality indication for each model, such as AICc, indicates a relative quality score compared to the other models for the data set. The quality indication may also be based on additional quality information. For example, parameter estimations and observations with “perfect predictions” may be determined. One or more of the quality measurements may provide the quality indication for each of the models and may be used to select a model to score and generate predictions. For example, the quality indication for each of the models maybe used to determine the best model or the model having the highest quality based on the quality indications. The selected model may be used to score the entire data set and generate predictions, e.g., confidence limits.

In another example, the modeling component 1326 may score each model generates and use a voting method to determine the predictions. Thus, the predictions may be based on an ensemble model of a plurality of models. The voting method includes determine predictions based on the agreement and/or disagreement of predictions generated by each of the plurality of models. For example, if three models are generated, each of the confidence limits may be based on the agreement of two or more of the models. More specifically, if two or three of the models agree on a lower confidence limit value for a particular event, e.g., DRG, that value is used for the lower confidence limit. If none of the models agree out of the then the confidence value in middle of the three predictions by the models may be used. Note that embodiments are not limited in this manner The confidence limit used may be based on a percentage of the models agreeing above a percentage threshold. For example, if six models were generated for a given data set to determine predictions for a target variable, the confidence limits used may be based on three or more of the models that are in agreement, e.g., equal to or greater than 50% threshold. The percentage threshold may be configurable based on a given set of data.

The model component 1326 may determine the predictions for the target variable, e.g., the expected length of stay, using one of the two above discussed methods. In the illustrated example, the predictions include a minimum and maximum prediction the length of stay for each DRG to create a range. In one specific example, the minimum and maximum predictions by DRG by patient are combined to create a range of predicted length of stay, such as 2(min)-6(max) days. Note that ranges may be specific for each event. For example, the range may be specific by DRG for each patient and predicted ranges are not the same on DRGs across patients, e.g., a patient may have a predicted range of 2-6 days for a particular DRG, but another patient could have a different predicted range for the same particular DRG. The variability of the lengths of stay for a particular DRG is a result automatically determining predictions across many DRGs and will cause variability in the confidence limits. The scored data set and the predictions may be stored in storage, and may be accessible to other components, such as the results component 1328. The results component 1328 may utilize the scored data set and predictions to score additional data sets, as will be discussed in more detail below.

In embodiments, a results component 1328 may use the one or more models to score a different data set. As similarly discussed, one of two methods may be used to score the data set. For example, the selected model indicating having the highest quality relative to the other model may be used to score the data when the predictions are generated based on this approach. The ensemble model and voting may be used to score the data set when that approach is utilized to generate the predictions. The predictions may be used to detect data outside of the predicted ranges, for example. This information can be used to identify anomalies, e.g., events associated with a timeframe outside of the predicted timeframe, and may indicate that further inspection is required.

In FIG. 19E, the results component 1328 obtains a data set to score based on the predictions generated from training. In embodiments, the data set may be obtained from one or more sources, such as an insurance company, a hospital, one or more public and/or private databases, and so forth. The results component 1328 may also score the data set including comparing the data in the data set with the predictions. For example, embodiments include comparing actual length of stays with predicted length of stays. The results component 1328 may also determine data outside of the predicted ranges, e.g., the actual length of stays outside of the predicted length of stays or actual maintenance timeframes outside of predicted timeframes. The data associated with the identified abnormal target variable may be flagged for further inspection. For example, at line 1987, the results component 1328 may provide results 1908 to a system that can further inspect abnormalities. Embodiments are not limited to these examples.

Embodiments discussed herein may also include the logic to generate the models and make predictions for a target variable. Other embodiments include a computer-implemented method, and/or at least one non-transitory computer-readable storage medium having instructions that when executed cause processing circuitry to perform the various operations discussed herein. These embodiments may provide technical advantages over previous systems by enabling a user of the system to interact with decision tree data structures to flag anomalies in real-time.

As discussed, some systems may use Hadoop®, an open-source framework for storing and analyzing big data in a distributed computing environment to generate models and probabilities of occurrence as discussed herein. Some systems may use cloud computing, which can enable ubiquitous, convenient, on-demand network access to a shared pool of configurable computing resources (e.g., networks, servers, storage, applications and services) that can be rapidly provisioned and released with minimal management effort or service provider interaction. Some grid systems may be implemented as a multi-node Hadoop® cluster, as understood by a person of skill in the art. Apache™ Hadoop® is an open-source software framework for distributed computing. Some systems may use the SAS® LASR™ Analytic Server in order to deliver statistical modeling and machine learning capabilities in a highly interactive programming environment, which may enable multiple users to concurrently manage data, transform variables, perform exploratory analysis, build and compare models and score with virtually no regards on the size of the data stored in Hadoop®. Some systems may use SAS In-Memory Statistics for Hadoop® to read big data once and analyze it several times by persisting it in-memory for the entire session.

What is claimed is:

1. An apparatus, comprising:
processing circuitry; and
memory to store instructions that, when executed by the processing circuitry, cause the processing circuitry to:
obtain patient data, the patient data comprising medical events, consideration received for medical events, length of stays for the medical events, and diagnosis related groups (DRGs) for the medical events;
determine a first subset of the patient data having consideration received for a medical event in a percentile grouping;
determine a second subset of the patient data having the consideration received in another percentile grouping;
generate a first model based on the first subset of the patient data, the first model for use to determine expected length of stay ranges for each of one or more DRGs;
generate a second model based on the second subset of the patient data, the second model for use to determine the expected length of stay ranges for each of one or more DRGs;
determine a first quality indication for the first model and a second quality indication for the second model, the first quality indication and the second quality indication based on one or more quality measurements, and the first quality indication and the second quality indication to indicate relative quality between the first model and the second model;
utilize the first quality indication and the second quality indication to select the first model or the second model having higher quality, the selected first model or second model to score the patient data; and
determine the expected length of stay ranges for the DRGs of the patient data based on the scoring of the patient data utilizing the selected first model or the second model, each of the expected length of stay ranges having a lower confidence limit and an upper confidence limit.

2. The apparatus of claim 1, the first and second quality indications based on one or more quality measurements comprising an Akaike Information Criterion-Corrected (AICc) measurement of the first model and the second model, output parameter estimates indicating DRGs having significance for the first model and the second model, a first count of predictions for the first model matching actual length of stays and a second count of predictions for the second model matching the actual length of stays.

3. The apparatus of claim 1, the processing circuitry to:
generate a third model based on the patient data, the third model for use to determine the expected length of stay ranges for each of one or more DRGs;
generate a third quality indication for the third model, the third quality indication based on one or more quality measurements of the third model;
utilize the third quality indication to select one of the first model, the second model, and the third model having higher quality, the selected first model, second model, or third model to score the patient data; and
determine the expected length of stay ranges for the DRGs of the patient data based on the scoring of the patient data utilizing the selected first model, the second model, or the third model.

4. The apparatus of claim 1, the processing circuitry to determine claims associated with length of stays outside of the expected length of stay ranges for each of the one or more DRGs.

5. The apparatus of claim 1, the processing circuitry to:
identify outlier length of stays in the patient data; and
remove patient data associated with the outlier length of stays from the first subset and the second subset prior to generating the first model and second model.

6. The apparatus of claim 1, the processing circuitry to identify locale information for the patient data and generate the first model and the second model based on the locale information.

7. The apparatus of claim 1, the processing circuitry to perform a log10 transformation on each length of stay in each of the first subset and the second subset prior to generating the first model and the second model.

8. The apparatus of claim 1, the processing circuitry to identify claims associated with a readmission within a period of time of a date of a current admission for each of the claims for use as a variable in generating the first model and the second model, and group correlated variables of the patient data into clusters to generate the first model and the second model.

9. The apparatus of claim 1, wherein each of the lower confidence limits is a minimum number of days and each of the upper confidence limits a maximum number of days.

10. The apparatus of claim 1, wherein the first model and the second model are generalized linear mixed models.

11. At least one non-transitory computer-readable storage medium comprising instructions that when executed cause processing circuitry to:
obtain patient data, the patient data comprising medical events, consideration received for medical events, length of stays for the medical events, and diagnosis related groups (DRGs) for the medical events;
determine a first subset of the patient data having consideration received for a medical event in a percentile grouping;
determine a second subset of the patient data having the consideration received in another percentile grouping;
generate a first model based on the first subset of the patient data, the first model for use to determine expected length of stay ranges for each of one or more DRGs;
generate a second model based on the second subset of the patient data, the second model for use to determine the expected length of stay ranges for each of one or more DRGs;
determine a first quality indication for the first model and a second quality indication for the second model, the first quality indication and the second quality indication based on one or more quality measurements, and the first quality indication and the second quality indication to indicate relative quality between the first model and the second model;
utilize the first quality indication and the second quality indication to select the first model or the second model having higher quality, the selected first model or second model to score the patient data; and
determine the expected length of stay ranges for the DRGs of the patient data based on the scoring of the patient data utilizing the selected first model or the second model, each of the expected length of stay ranges having a lower confidence limit and an upper confidence limit.

12. The non-transitory computer-readable storage medium of claim 11, the first and second quality indications based on one or more quality measurements comprising an Akaike Information Criterion-Corrected (AICc) measurement of the first model and the second model, output parameter estimates indicating DRGs having significance for the first model and the second model, a first count of predictions for the first model matching actual length of stays and a second count of predictions for the second model matching the actual length of stays.

13. The non-transitory computer-readable storage medium of claim 11, comprising instructions that when executed cause the processing circuitry to:
generate a third model based on the patient data, the third model for use to determine the expected length of stay ranges for each of one or more DRGs;
generate a third quality indication for the third model, the third quality indication based on one or more quality measurements of the third model;
utilize the third quality indication to select one of the first model, the second model, and the third model having higher quality, the selected first model, second model, or third model to score the patient data; and
determine the expected length of stay ranges for the DRGs of the patient data based on the scoring of the patient data utilizing the selected first model, the second model, or the third model.

14. The non-transitory computer-readable storage medium of claim 11, comprising instructions that when executed cause the processing circuitry to determine claims associated with length of stays outside of the expected length of stay ranges for each of the one or more DRGs.

15. The non-transitory computer-readable storage medium of claim 11, comprising instructions that when executed cause the processing circuitry to:
identify outlier length of stays in the patient data; and
remove patient data associated with the outlier length of stays from the first subset and the second subset prior to generating the first model and second model.

16. The non-transitory computer-readable storage medium of claim 11, comprising instructions that when executed cause the processing circuitry to identify locale information for the patient data and generate the first model and the second model based on the locale information.

17. The non-transitory computer-readable storage medium of claim 11, comprising instructions that when executed cause the processing circuitry to perform a log10 transformation on each length of stay in each of the first subset and the second subset prior to generating the first model and the second model.

18. The non-transitory computer-readable storage medium of claim 11, comprising instructions that when executed cause the processing circuitry to identify claims associated with a readmission within a period of time of a date of a current admission for each of the claims for use as a variable in generating the first model and the second model, and group correlated variables of the patient data into clusters to generate the first model and the second model.

19. The non-transitory computer-readable storage medium of claim 11, wherein each of the lower confidence limits is a minimum number of days and each of the upper confidence limits a maximum number of days.

20. The non-transitory computer-readable storage medium of claim 11, wherein the first model and the second model are generalized linear mixed models.

21. A computer-implemented method, comprising:
obtaining patient data, the patient data comprising medical events, consideration received for medical events, length of stays for the medical events, and diagnosis related groups (DRGs) for the medical events;
determining a first subset of the patient data having consideration received for a medical event in a percentile grouping;
determining a second subset of the patient data having the consideration received in another percentile grouping;
generating a first model based on the first subset of the patient data, the first model for use to determine expected length of stay ranges for each of one or more DRGs;
generating a second model based on the second subset of the patient data, the second model for use to determine the expected length of stay ranges for each of one or more DRGs;
determining a first quality indication for the first model and a second quality indication for the second model, the first quality indication and the second quality indication based on one or more quality measurements, and the first quality indication and the second quality indication to indicate relative quality between the first model and the second model;
utilizing the first quality indication and the second quality indication to select the first model or the second model having higher quality, the selected first model or second model to score the patient data; and
determining the expected length of stay ranges for the DRGs of the patient data based on the scoring of the patient data utilizing the selected first model or the second model, each of the expected length of stay ranges having a lower confidence limit and an upper confidence limit.

22. The computer-implemented method of claim 21, the first and second quality indications based on one or more quality measurements comprising an Akaike Information Criterion-Corrected (AICc) measurement of the first model and the second model, output parameter estimates indicating DRGs having significance for the first model and the second model, a first count of predictions for the first model matching actual length of stays and a second count of predictions for the second model matching the actual length of stays.

23. The computer-implemented method of claim 21, comprising:
generating a third model based on the patient data, the third model for use to determine the expected length of stay ranges for each of one or more DRGs;
generating a third quality indication for the third model, the third quality indication based on one or more quality measurements of the third model;
utilizing the third quality indication to select one of the first model, the second model, and the third model having higher quality, the selected first model, second model, or third model to score the patient data; and
determining the expected length of stay ranges for the DRGs of the patient data based on the scoring of the patient data utilizing the selected first model, the second model, or the third model.

24. The computer-implemented method of claim 21, comprising determining claims associated with length of stays outside of the expected length of stay ranges for each of the one or more DRGs.

25. The computer-implemented method of claim 21, comprising:
identify outlier length of stays in the patient data; and
removing patient data associated with the outlier length of stays from the first subset and the second subset prior to generating the first model and second model.

26. The computer-implemented method of claim 21, comprising identifying locale information for the patient data and generate the first model and the second model based on the locale information.

27. The computer-implemented method of claim 21, comprising performing a log10 transformation on each length of stay in each of the first subset and the second subset prior to generating the first model and the second model.

28. The computer-implemented method of claim 21, comprising identifying claims associated with a readmission within a period of time of a date of a current admission for each of the claims for use as a variable in generating the first model and the second model, and group correlated variables of the patient data into clusters to generate the first model and the second model.

29. The computer-implemented method of claim 21, wherein each of the lower confidence limits is a minimum number of days and each of the upper confidence limits a maximum number of days.

30. The computer-implemented method of claim 21, wherein the first model and the second model are generalized linear mixed models.
